(19) United States
(12) Patent Application Publication
(10) Pub. No.: US 2026/0266810 A1
(43) Pub. Date: Sep. 10, 2026

(54) A METHOD FOR DETECTING AN ANALYTE

(71) Applicant: Psyros Diagnostics Limited, Sandwich Kent (GB)

(72) Inventors: Steven Andrew Ross, Aldington Kent (GB); Mark Thomas Gatton Swayne, Herne Bay, Kent (GB); Aileen Jane McGettrick, Herne Bay, Kent (GB); Paul Brendan Monaghan, Sandwich Kent (GB); Julie Richards, Faversham Kent (GB)

(21) Appl. No.: 19/166,439

(22) PCT Filed: Mar. 15, 2024
(86) PCT No.: PCT/GB2024/050714

§ 371 Date: Sep. 17, 2025

(30) Foreign Application Priority Data
Mar. 17, 2023 (GB) 2303924.1

(51) Int. Cl. G01N 33/543; G01N 33/58

(52) U.S. Cl. CPC G01N 33/54306; G01N 33/54366; G01N 33/582

(57) ABSTRACT

The present invention provides a method for detecting two or more analytes in a sample, the method comprising steps (i)-(v). Thus, the present invention provides a method for detecting two or more analytes in a sample in which only the reporter reagents in the vicinity of the surface of the substrate results in a signal, the signal being a local region of the optical component in the second optical state. It is the set of local regions of the optical component in the second optical state that are detected.

The present invention relates to a method for detecting an analyte, and particularly to a method for detecting two or more analytes in a sample.

There are many techniques available for the measurement of biologically relevant parameters in human samples, such as blood, plasma, serum, tissue etc. A common method is to use a capture reagent that binds to the target of interest, and a reporter reagent that has a label of some sort. The capture reagent can be bound to a solid phase, such as a microtitre plate, a bead or a membrane. When the sample is incubated with the capture reagent, the analyte binds to the capture reagent. The reporter reagent also binds to the analyte. Excess reporter can then be removed (by washing) and the quantity of reporter reagent can be measured, thus giving a measure of the amount of analyte present in the sample. There are many different variations on how these types of binding assay can be carried out. For example, the analyte can bind to the capture reagent first, then the reporter can be added in a separate step, or the analyte can bind to the reporter first, and then bind to the capture reagent.

There are a wide range of reagents that can be used as capture and reporter in binding assays of this type, including nucleic acids, carbohydrates, antigens, peptides, proteins and antibodies. There are also a wide range of target analytes, including peptides, proteins, antibodies, nucleic acids, cells, carbohydrates, small molecules, therapeutic drugs, drugs of abuse, steroids, hormones, lipids etc.

Assays that use antibodies are commonly termed immunoassays. Immunoassays can take a number of formats. For example, when a capture antibody is used to capture the analyte and a reporter antibody is used to generate a measurable signal, this is commonly called a sandwich immunoassay. Alternative formats are known, where the binding agent is attached to a solid phase and target analyte competes in solution with a labelled reagent that also binds to the binding agent. In the absence of analyte, a high signal is obtained, since high levels of the labelled reagent bind. In the presence of analyte, a fraction of the binding sites are blocked, thus less of the labelled reagent binds and the signal is reduced. These assays are commonly known as inhibition or competition assays. Several types of competition assay are known. For example, an antibody could be bound to the solid phase and labelled analyte (or analogue of the analyte) can compete for the binding sites on the antibody. Alternatively, an analogue of the analyte could be immobilised, and a labelled antibody could bind to this surface. In the presence of analyte in the sample, this will bind to the antibody in solution, preventing binding to the surface, and signal will be diminished.

There are many formats for assays, and many different types of label that can be employed. For example, assays can be heterogeneous, in that excess label is removed before the measurement takes place, for example by the use of wash steps. Removal of excess label can also be achieved by flowing sample and reporter past a capture area. This approach is used in immunochromatographic or lateral flow strips, which are used in rapid testing for infectious disease tests and pregnancy tests, for example. Alternatively, homogeneous assays are known, where the excess reporter is not removed. Homogeneous assays tend to rely on the close proximity of capture and reporter creating some form of signal. One example of a homogeneous assay is an agglutination assay, where particles bind together in solution. The agglutinated particles cause scattering of light, which can be measured by turbidimetry or nephelometry. A further example of a homogeneous assay using particles is the luminescent oxygen channeling immunoassay (LOCI), described in further detail below.

Another example of a homogeneous assay is the fluorescence resonance energy transfer (FRET), where the capture and reporter reagents are donor and acceptor fluorophores respectively, and excitation of the donor leads to energy transfer to the acceptor, followed by light emission.

One homogeneous assay format that functions in whole blood, without removal of cellular material is the pyro-optical immunoassay. Capture antibody is coated on to pyroelectric polyvinylidene PVDF sensor and carbon particles are used as the reporter. Signal is generated by illuminating the sample with light, causing localised heating of the particles. Those bound to the sensor transfer energy to the pyroelectric sensor, causing a thermal stress that is detected as an electrical signal. The more carbon bound, the greater the signal.

The label that is attached to the reporter binding agent can be an agent that absorbs light, such as a dye, gold particle or dyed latex microsphere. Larger particles can, in principle, absorb more light, and generate more signal. However, there is a size limit where particulate labels become impractical to use in assays, as described in more detail below. Luminescent labels are also known, such as fluorescent, chemiluminescent, bioluminescent and electrochemiluminescent labels. Luminescent labels have also been encapsulated in particles for certain types of assay. Amplification of the signal may also be performed by using enzymatic or catalytic reactions. Enzymes can be used to convert a substrate from a leuco dye to a coloured form, or into a fluorescent or luminescent form. It is common for the excess enzyme to be removed using wash steps before the substrate is added, so that signal is generated only by the enzyme that is bound specifically to the analyte.

Immunoassays that do not use labels are also known, such as assays that use surface plasmon resonance as the signal transduction method. However, label free assays tend to lack the sensitivity of assays that use labels to enhance the signal.

Further information on the field of immunoassays can be found in “The Immunoassay Handbook: 4th Edition: Theory and Applications of Ligand Binding, ELISA and Related Techniques”, Ed. D. Wild, Elsevier Science, 2013.

All binding assays, including immunoassays, have limitations in terms of the minimum and maximum concentrations of analyte that can be reliably measured.

The signal maximum is generally limited by factors such as the total amount of capture antibody available to bind the analyte and the total amount of reporter antibody to generate signal. If the capture antibody is immobilised on a solid-phase, then the surface area of the solid-phase can limit the upper limit of detection. Additionally, some signal transduction techniques can be prone to saturation, such as colorimetric methods, depending on the pathlength that the light needs to take through the sample. Luminescent methods are less prone to saturation, as the gain on the detector can be attenuated to deal with higher levels of light emission. When all of the antibody binding sites in a heterogeneous assay are filled with analyte, then the maximum signal is reached and the system has saturated. The excess analyte will normally be removed in a wash step before the reporter is added. Homogeneous assays can also suffer from an effect known as high-dose hook, where the concentration of analyte is greater than the effective concentration of capture and/or reporter antibody. In this instance, at very high concentrations, all of the binding sites on the capture and the reporter can be blocked and the assay signal can be diminished, giving erroneous results.

The lower level of detection is governed by a number of different factors. Generally, all assays will be affected by attributes such as the quality of the antibodies being used (affinity and specificity) and cross-reactivity of the antibodies with related analytes. The lower limit of detection is also dependent upon the assay set-up and factors which affect the signal-to-noise ratio of the system design. For example, in a standard enzyme-linked immunosorbent assay (ELISA), capture antibody is coated onto the surface of a 96-well microtitre plate, and then sample is incubated in the wells, leading to the analyte being captured. The wells are washed, and then reporter is added in excess, binding to the captured analyte. Excess reporter is then washed away and substrate is added that can react with the enzyme, being converted into an active form. For example, a non-coloured leuco dye, such as 2,2′-azino-bis(3-ethylbenzothiazoline-6-sulphonic acid) (ABTS) can be converted by horseradish peroxidase into an oxidised green form in the presence of hydrogen peroxide. When the analyte is present in very low quantities (e.g. less than 1 picomolar), there will only be very small quantities of enzyme bound to the surface of the well. The ABTS reacts with the enzyme to generate the green form, then diffuses into the bulk of the fluid, generating a solution that is so dilute that it cannot be distinguished from background signal. Auto-conversion of substrate can also generate colour that interferes with the measurement. Similarly, other detection methods, such as fluorescence can suffer from interfering factors and auto-fluorescence of components in the sample or the reaction wells.

Another confounding factor in immunoassays can be non-specific binding of reporter reagents to the capture surface. For example, in the ELISA assay described above, the microtitre well is coated in a layer of protein, some of which can become de-natured during the coating process. It is not uncommon for reporter to bind to areas of the capture surface during the assay. If this reporter turns over substrate to contribute to the overall signal, it is not possible to distinguish the signal due to specifically bound reporter from that which is bound non-specifically. Non-specific binding can also be promoted by many of the components present in the original sample, which can bind to the capture surface during the initial incubation, modifying the surface properties of the capture layer, creating a surface that can bind the reporter. Minimising non-specific binding of reporter involves careful optimisation of all reagents and reaction conditions used during the assay, including antibodies, detergents, temperature and ionic strength.

Generally, the limit of detection of traditional immunoassays is around 0.1 picomolar to 1 nanomolar, depending upon the assay methodology. Developing assays with very low detection limits using traditional approaches often requires a great deal of optimisation, with stringent wash steps to reduce non-specific binding and maximise signal-to-noise. Additionally, the capture surface is often small in relation to the sample volume to ensure that the signal is high enough relative to the background.

One approach that has been used to circumvent issues relating to signal-to-noise and improve detection limits is to measure individual binding events, and to count these binding events as “on” or “off” events, if the measurement is above a localised threshold. In this way, much of the background noise can be eliminated. Analogies can be drawn with digitisation of audio or communication signals. These digital assays have been shown to reach detection limits that were previously unattainable using traditional analogue methods. For example, detection limits in the low femtomolar (10⁻¹⁵ mol/L) and even attomolar (10⁻¹⁸ mol/L) range have been reported. See for example “Evaluation of highly sensitive immunoassay technologies for quantitative measurements of sub-pg/mL levels of cytokines in human serum”, Yeung et al., Journal of Immunological Methods, 2016, 437, 53 and “Digital Detection of Biomarkers Assisted by Nanoparticles: Application to Diagnostics”, Cretich et al., Trends in Biotechnology, 2015, 33, 343.

The majority of labels/reporters used in assays (such as fluorophores, dyes etc.) are not visible individually using wide-field microscopy, even under high magnification, as their size is below the diffraction limit of visible light. Thus, the presence of these labels can only be measured as a bulk phenomenon, not by counting each label. In contrast, particulate labels, such as latex particles, can in theory be visualised by wide-field optical microscopy if they are above a certain size. Depending upon the optical set-up, it is possible to start visualising particles when they have a diameter of several hundred nanometres and above, depending on the numerical aperture and type of microscope.

However, using particles of this size as labels to monitor individual binding events on a capture surface (such as an antibody-antigen interaction) becomes impractical for a number of reasons. For example, particles of this size diffuse very slowly in comparison to other types of label, impairing the rate of the reaction at a planar surface. They also start to exhibit macroscopic buoyancy effects and will sediment or float if the density of the particle is significantly different to the medium that they are contained in, which can also cause issues with assay format. Particles of this size are particularly prone to binding non-specifically to surfaces, causing high background, which is difficult to remove. Finally, excess particles must be removed, requiring wash steps. However, large particles start to suffer from shear effects in the presence of fluid flow, where the shear force on the particle becomes greater than the rupture strength of the antibody-antigen interaction (approximately 60-250 pN), leading to particles being washed away (see “Rapid Femtomolar Bioassays in Complex Matrices Combining Microfluidics and Magnetoelectronics”, Mulvaney et al., Biosensors and Bioelectronics, 2007, 23, 191).

Examples of digital assays include the Quanterix Single Molecule Array (SIMOA) system and the Merck Millipore Single Molecule Counting (SMC) system.

The Quanterix SIMOA system uses antibody-coated paramagnetic beads to capture analyte from solution. The magnetic beads are then washed, and reporter antibody labelled with enzyme is added. The quantity of beads is sufficient that the probability of having more than one analyte and reporter per bead is minimised. The beads are washed again, then loaded into an array of microwells that can only hold one bead per well. The volume of the microwells is on the femtolitre scale. If the bead has enzyme attached, then fluorogenic substrate in the well is turned over. The small dimensions of the well prevent the fluorescent product from becoming too diffuse. Each well is then counted as an “on” or “off” event if the fluorescence is above a threshold value.

The SMC system is used in the Erenna and SMCxPRO systems from Merck Millipore. The fundamental measurement technique in both systems is the same. Magnetic beads, coated in capture antibody, are used to capture the target analyte in a sandwich assay. Fluorescently labelled reporter antibody also binds to the bead, in the presence of analyte. The beads are pulled down with a magnet and excess fluorescently-tagged reporter is washed away. An eluting buffer is then added that causes dissociation of the sandwich complex, which is then transferred to a measurement vessel. The presence of the fluorescent tag is then measured using a confocal fluorescent microscope that sequentially interrogates small volumes of the sample to determine if the fluorescent tag is present or not. If the signal for each individual measurement is above a threshold value, this counts as an “on” event for that measurement.

Several independent academic reviews of high-sensitivity immunoassays have highlighted that the digital approach to immunoassays allows unprecedented improvements to detection limits (see references Yeung and Cretich above).

The detection limit of the Quanterix and Merck Millipore systems is dependent upon the volume of sample that is used in the assay. For a 10 microlitre serum or plasma sample, the theoretical limit would be the detection of a single binding event, corresponding to 1 molecule. However, in terms of molarity, this would correspond to 100,000 molecules per litre of sample, or 0.16×10-18 moles per litre (0.16 attomolar).

However, the Quanterix and Merck Millipore systems described above are complex and cumbersome, each requiring a number of wash and transfer steps. Further, the assays can only be carried out for samples that do not contain cellular material and the systems require expensive instrumentation in order to achieve the performance that they offer. There therefore remains a need for simpler and more cost-effective high-sensitivity systems.

Additionally, the Quanterix and Merck Millipore systems measure one analyte per sample. Measurement of multiple analytes in the same sample in a homogeneous assay format is usually achieved by one of two approaches. The first approach uses separate reporter reagents for each analyte and separate physical areas containing binding components (capture reagents) that bind each reporter reagent in the presence of analyte. Therefore, this approach achieves multiple measurements by spatial separation. However, if you have five analytes, five reporting reagents and five binding components (capture reagents), there is the potential for 80% of reporter reagent at each measurement area to be unwanted reporter reagent that could interfere with the measurement. The second approach uses optical labels that can be visualised at separate wavelengths. However, this approach necessitates the use of electromagnetic radiation at different wavelengths, which requires multiple light sources and/or complex optical filters, diffraction gratings or the like.

WO2020/260865 describes a digital assay method that uses a photosensitiser reagent to convert an optical component from a first optical state to a second optical state. However, there remains a need for optimising this system to measure multiple analytes simultaneously (multiplexing) without added complexity or interference between each measurement.

Accordingly, the present invention provides a method for detecting two or more analytes in a sample, the method comprising the steps of:
(i) providing a mixture comprising a sample and two or more reporter reagents to a device, wherein each of the two or more reporter reagents comprises a photosensitiser, the device comprising a substrate having an optical component and two or more binding components, wherein the optical component and the two or more binding components are attached to a surface of the substrate;
(ii) allowing a portion of each of the two or more reporter reagents to bind to the surface of the substrate in proportion to the concentration of the corresponding analyte, by means of the corresponding binding component;
(iii) irradiating the device with electromagnetic radiation at one wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) optionally detecting the at least one set of local regions of the optical component having the second optical state on the substrate formed in step (iii) and repeating step (iii) with electromagnetic radiation at the same or a different wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from the first optical state to the second optical state, thereby forming at least one set of local regions of the optical component having the second optical state on the substrate; and
(v) if step (iv) is performed, detecting the at least one set of local regions of the optical component having the second optical state on the substrate formed in step (iv) or, if step (iv) is not performed, detecting two or more sets of local regions having the second optical state on the substrate formed in step (iii),
wherein the two or more sets of local regions are distinguishable from one another by rate of formation of the sets of local regions, order of formation of the sets of local regions, or size of local regions optionally in combination with rate of formation of the sets of local regions or order of formation of the sets of local regions.

Thus, the present invention provides a method for detecting two or more analytes in a sample in which only the reporter reagents in the vicinity of the surface of the substrate results in a signal, the signal being a local region of the optical component in the second optical state. It is the set of local regions of the optical component in the second optical state that are detected. Therefore, the present invention simplifies digital detection of two or more analytes and facilitates homogeneous assays on a range of samples, including those containing cellular materials.

The fact that there are two or more sets of local regions that are distinguishable from one another by rate of formation of the sets of local regions, order of formation of the sets of local regions, or size of local regions optionally in combination with rate of formation of the sets of local regions or order of formation of the sets of local regions provides a simple method for detecting and distinguishing between two or more analytes in a sample.

In contrast to typical methods for measuring two or more analytes in a sample in a homogeneous assay format, there is no requirement in the method of the present invention to separate the two or more binding components on the surface of the substrate and no requirement to use electromagnetic radiation at different wavelengths to detect two or more analytes in a sample.

However, if the two or more binding components on the surface of the substrate are separated into different areas, specific binding of the reporter reagent expected to bind and non-specific binding of unwanted reporter reagent can be distinguished because the two or more sets of local regions are distinguishable from one another by rate of formation of the sets of local regions, order of formation of the sets of local regions, or size of local regions optionally in combination with rate of formation of the sets of local regions or order of formation of the sets of local regions. This reduced interference and increased accuracy of the method of the present invention is a significant advantage over multiplexed assays that add all the different reporter reagents to one sample and then distinguish binding solely by varying the binding component by location.

Therefore, the present invention provides an improved method for detecting two or more analytes in a sample.

The present invention will now be described with reference to the drawings, in which:

FIG. 1 shows various components which may be used in the method of the present invention;

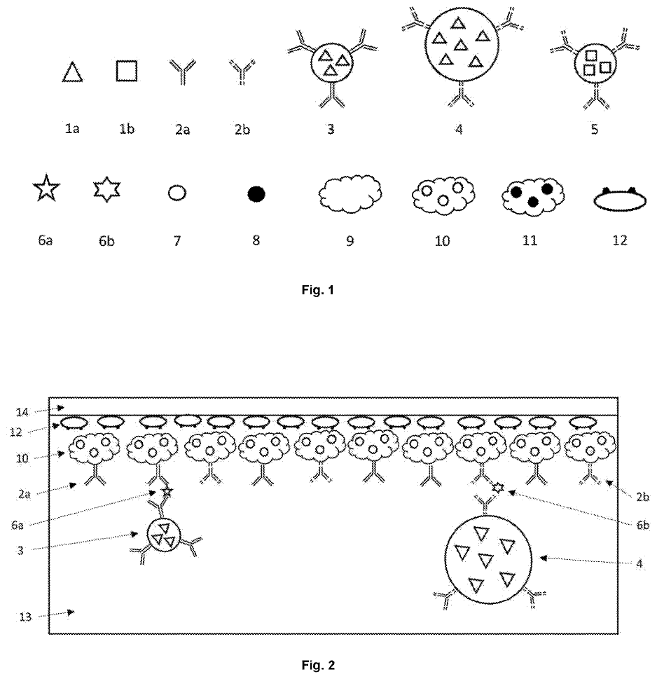

FIG. 2 shows a device in which two reporter reagents of different sizes are bound to the surface of the substrate before irradiation;

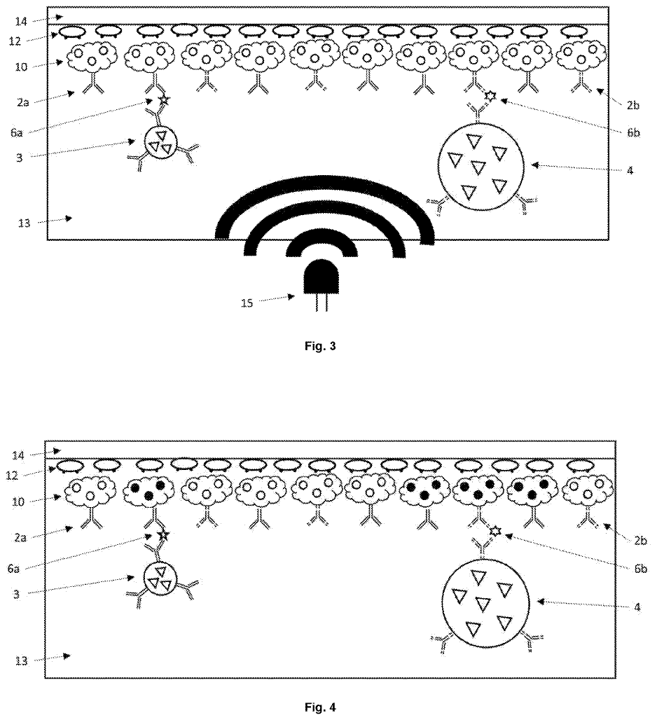

FIG. 3 shows the device of FIG. 2 being irradiated;

FIG. 4 shows the device of FIG. 3 after irradiation;

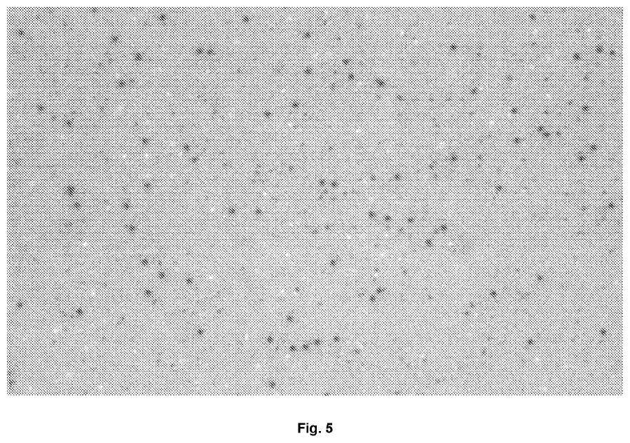

FIG. 5 shows a representative example of two sets of local regions having the second optical state on the substrate, wherein the two sets of local regions are distinguishable from one another by size of local regions, visible as differently sized dark areas bleached in a fluorescent layer;

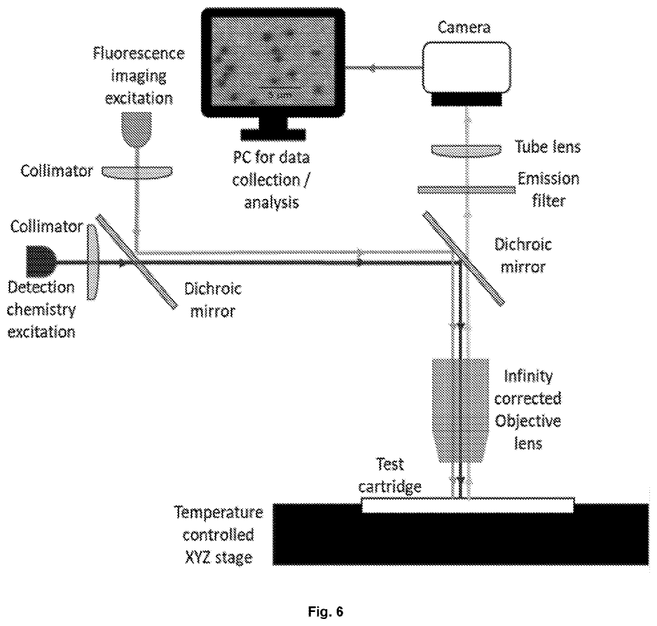

FIG. 6 shows an optical set-up for detection that allows two electromagnetic radiation sources to be focused through an objective lens;

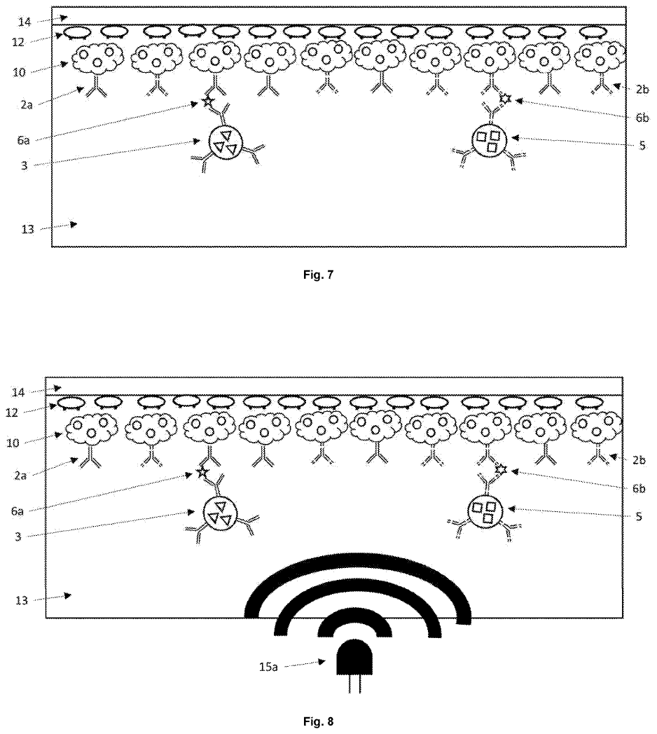

FIG. 7 shows a device in which two reporter reagents comprising different photosensitisers are bound to the surface of the substrate before irradiation;

FIG. 8 shows the device of FIG. 7 being irradiated at wavelength a;

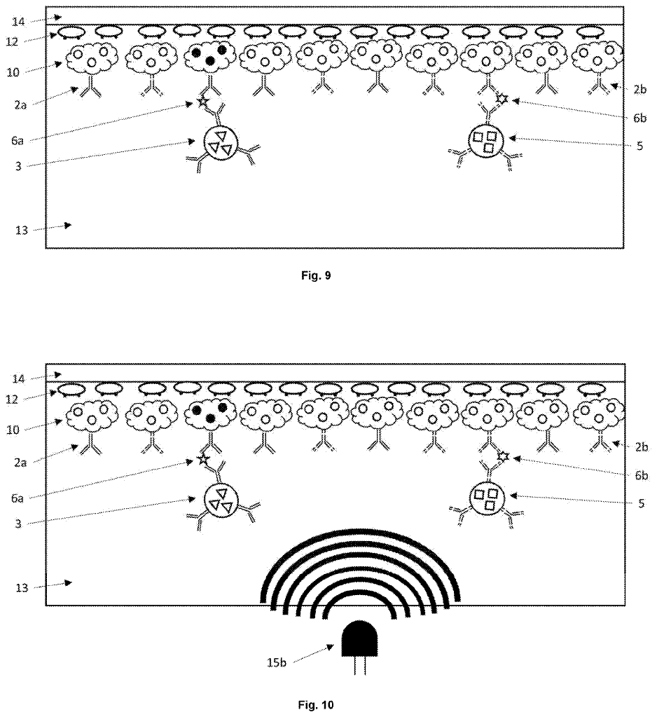

FIG. 9 shows the device of FIG. 8 after irradiation at wavelength a;

FIG. 10 shows the device of FIG. 9 being irradiated at wavelength b;

FIG. 11 shows the device of FIG. 10 after irradiation at wavelength b;

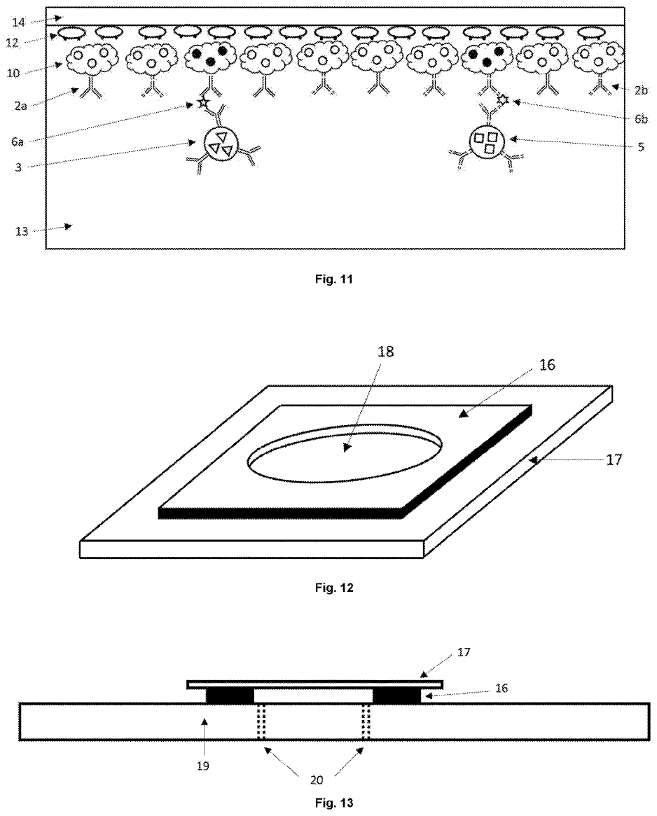

FIG. 12 shows a substrate and well created for the method of the present invention;

FIG. 13 shows a sample chamber created using the substrate and well of FIG. 12;

FIG. 14 shows a device in which each of the two binding components is attached to a separate area of the substrate (binding component 2a in well 1 and binding component 2b in well 2) and the two reporter reagents of different sizes are bound to the surface of the substrate before irradiation;

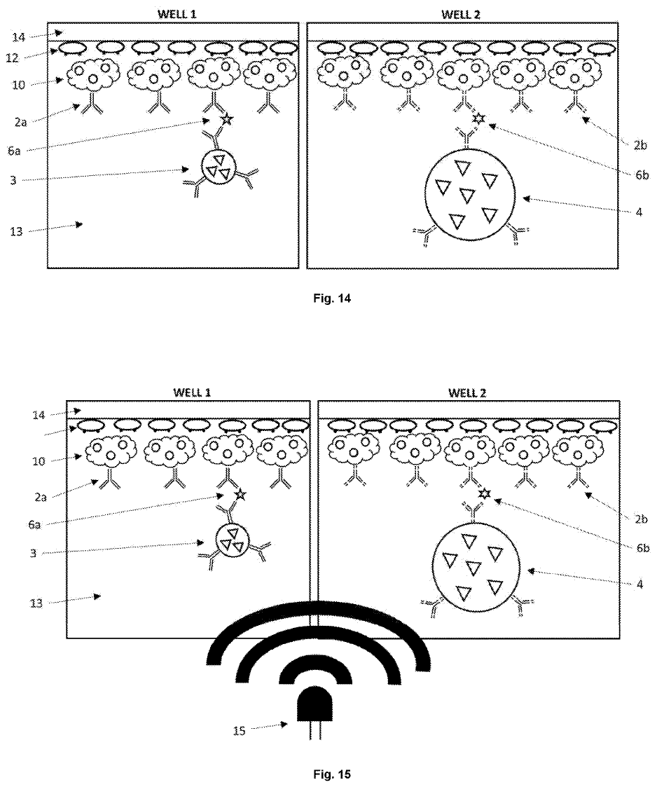

FIG. 15 shows the device of FIG. 14 being irradiated;

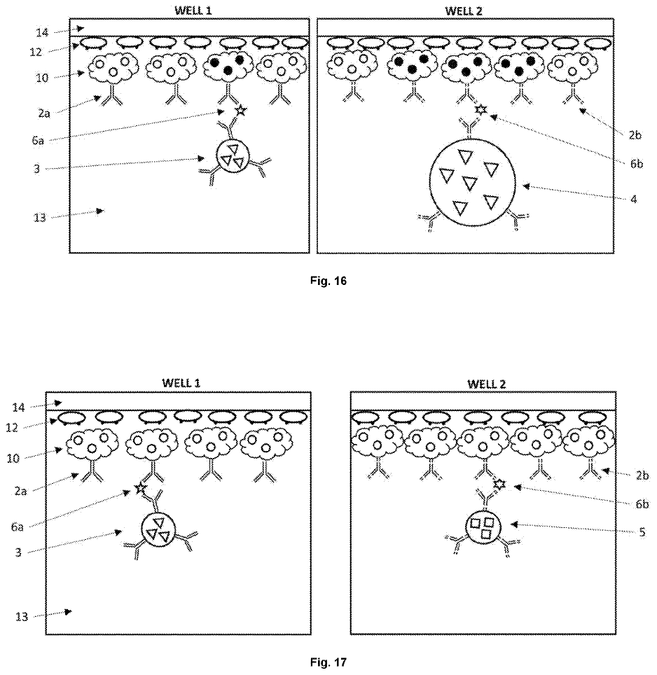

FIG. 16 shows the device of FIG. 15 after irradiation;

FIG. 17 shows a device in which each of the two binding components is attached to a separate area of the substrate (binding component 2a in well 1 and binding component 2b in well 2) and the two reporter reagents comprising different photosensitisers are bound to the surface of the substrate before irradiation;

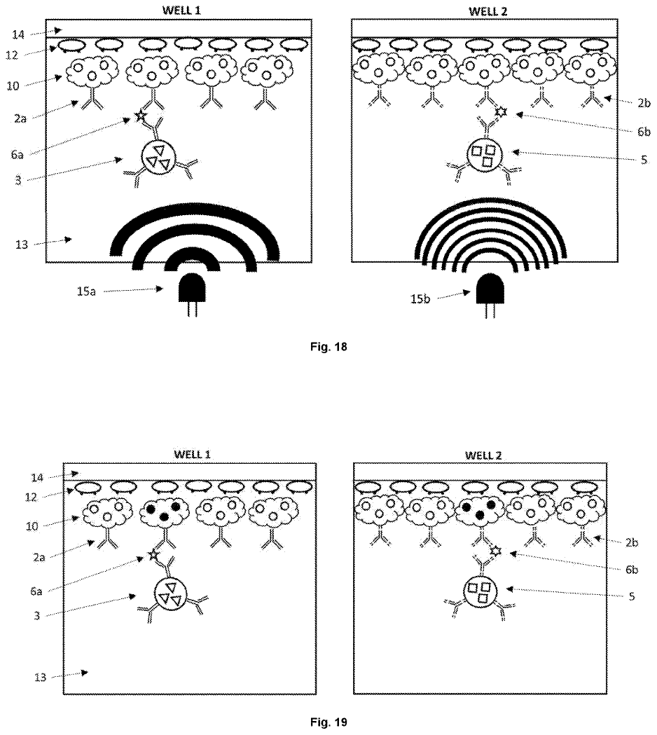

FIG. 18 shows the device of FIG. 17 being irradiated; and

FIG. 19 shows the device of FIG. 18 after irradiation.

The method of the present invention is used for detecting two or more analytes in a sample (which may be via the detection of a complex or derivative of the analyte). The two or more analytes are different types of analyte to form two or more sets of local regions having the second optical state on the substrate. The method of the present invention is therefore used to detect multiple analytes simultaneously (multiplexing). There is no theoretical upper limit on the number of analytes that can be detected but the number of analytes that can be detected is likely to be constrained by the practicalities of being able to reliably distinguish different sets of local regions.

In a preferred embodiment, the method of the present invention is a method for detecting two to ten analytes in a sample, more preferably two to eight analytes in a sample, more preferably two to six analytes in a sample and most preferably two to four analytes in a sample.

The components of FIG. 1 are: photosensitisers 1a and 1b which are stimulated by different wavelengths of radiation; antibodies 2a and 2b which bind to different analytes; antibody-coated latex particle 3, infused with photosensitiser 1a (also referred to below as photosensitiser-labelled antibody 3); antibody-coated latex particle 4, which is different in size to particle 3 but still infused with photosensitiser 1a (also referred to below as photosensitiser-labelled antibody 4); antibody-coated latex particle 5, infused with photosensitiser 1b (also referred to below as photosensitiser-labelled antibody 5); protein analytes 6a and 6b; optical component in fluorescent state 7; optical component in non-fluorescent state 8; streptavidin 9, streptavidin labelled with optical component in fluorescent state 10 (also referred to below as streptavidin-dye conjugate 10); streptavidin labelled with optical component in non-fluorescent state 11; and biotinylated BSA 12 (also referred to below as BSA-biotin conjugate 12).

Step (i) of the method of the present invention involves providing a mixture comprising a sample and two or more reporter reagents to a device, wherein each of the two or more reporter reagents comprises a photosensitiser, the device comprising a substrate 14 having an optical component and two or more binding components, wherein the optical component and the two or more binding components are attached to a surface of the substrate 14.

The sample and the two or more reporter reagents may be pre-mixed before adding the mixture to the device or the sample and the two or more reporter reagents may be added sequentially to the device to form the mixture. The mixture typically contains additional reagents such as buffer, detergent and other additives.

Each of the two or more reporter reagents corresponds to a different analyte and binding component. For example, a first reporter reagent corresponds to a first analyte and a first binding component, a second reporter reagent corresponds to a second analyte and a second binding component, and so on.

By way of an explanation of the principle underlying the present invention, FIG. 2 shows a device in which two reporter reagents are bound to the surface of the substrate before irradiation. The device includes a substrate 14 and a sample chamber 13 for holding a sample containing protein analytes 6a and 6b dissolved or suspended therein. The substrate may be any substrate that allows detection of the two or more sets of local regions having the second optical state on the substrate. Preferably, the substrate is planar. Preferably, the substrate is a transparent substrate and more preferably, the substrate is glass or plastic.

The substrate 14 has antibodies 2a and 2b bound to streptavidin-dye conjugate 10 attached to a surface of the substrate 14 via BSA-biotin conjugate 12. The dye acts as the optical component and the antibodies act as the binding components. BSA-biotin conjugate 12 is an inert macromolecule that facilitates attachment of the optical and binding components to the surface of the substrate 14.

Although the optical and binding components are shown in this way, any technique for holding the optical and binding components proximal to the surface of the substrate 14 is applicable. For example, the optical and binding components may be a single reagent. The optical component may also be encapsulated within a polymer layer which is coated onto the surface of the substrate 14 and the binding components attached to the polymer layer. The polymer may be silicone, polystyrene or polyisobutylene, or any other suitable polymeric plastic that can be used to encapsulate the optical component.

Alternatively, a gel layer, e.g. a hydrogel layer may be impregnated with the optical component and the gel/hydrogel layer coated onto the surface of the substrate 14 and the binding components attached to the gel/hydrogel layer.

Step (ii) of the method of the present invention involves allowing a portion of each of the two or more reporter reagents to bind to the surface of the substrate in proportion to the concentration of the corresponding analyte, by means of the corresponding binding component. As discussed above, each of the two or more reporter reagents corresponds to a different analyte and binding component. For example, a first reporter reagent corresponds to a first analyte and a first binding component, a second reporter reagent corresponds to a second analyte and a second binding component, and so on. Therefore, by corresponding analyte is meant the analyte specific to the reporter reagent and by corresponding binding component is meant the binding component specific to the reporter reagent.

Step (ii) may be achieved by allowing the device to stand for a period of time, for example 10 minutes.

In FIG. 2, photosensitiser-labelled antibody 3 is bound to the surface of the substrate by means of antibody 2a and photosensitiser-labelled antibody 4 is bound to the surface of the substrate by means of antibody 2b. Photosensitiser-labelled antibodies 3 and 4 act as the reporter reagents.

The two or more reporter reagents bind to the surface of the substrate in proportion to the concentration of the corresponding analyte. For example, if the two or more binding components and the two or more reporter reagent are antibodies, and the two or more analytes are antigens, each of the two or more reporter reagents bind to the corresponding binding component via the corresponding analyte to form a so-called “sandwich” complex. In FIG. 2, photosensitiser-labelled antibody 3 binds to antibody 2a via protein analyte 6a. Similarly, photosensitiser-labelled antibody 4 binds to antibody 2b via protein analyte 6b. Other binding events, such as antibody-hapten binding or nucleic acid binding are also possible.

All steps up until this point have taken place in the absence of light. Step (iii) of the method of the present invention involves irradiating the device with electromagnetic radiation at one wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from a first optical state to a second optical state, thereby forming at least one set of local regions of the optical component having the second optical state on the substrate 14.

FIG. 3 shows the device of FIG. 2 being irradiated with electromagnetic radiation, preferably visible light. The light source may be, for example, LED 15. The light source illuminates the sample chamber 13 with light of the appropriate wavelength to excite the photosensitiser 1a of photosensitiser-labelled antibodies 3 and 4.

The wavelength depends on the photosensitiser. The device is typically irradiated for 10 seconds. Preferably, the device is irradiated with electromagnetic radiation for 1 to 30 seconds, more preferably 2 to 20 seconds, more preferably 5 to 15 seconds and most preferably 10 seconds. This ensures that there is an irreversible optical change on the surface of the substrate, thus allowing long-lived and transient binding events to be distinguished.

FIG. 4 shows the device of FIG. 3 after irradiation. The photosensitiser 1a of photosensitiser-labelled antibodies 3 and 4 interacts with the dye optical component of the streptavidin-dye conjugate 10 to cause the dye to change from a fluorescent state to a non-fluorescent state. Streptavidin-dye conjugate in fluorescent state 10 becomes streptavidin-dye conjugate in non-fluorescent state 11. Only the dye that is in close proximity to photosensitiser 1a changes from the first optical state to the second optical state.

Alternative first and second optical states could include changes to light polarisation, fluorescence lifetime, refractive index, light scattering (including Raman scattering), phosphorescence and other optical effects.

Although FIGS. 2-4 illustrate one of each of the two reporter reagents bound to the surface of the substrate 14, in reality a number of the same reporter reagent will bind to the surface of the substrate 14 to create more than one local region having the second optical state on the substrate in a set of local regions having the second optical state on the substrate. Using the components of FIG. 1, these local regions having the second optical state on the substrate would be visible as discrete areas with the streptavidin-dye conjugate in non-fluorescent state 11.

Such discrete areas can be seen in FIG. 5, which shows a representative example of two sets of local regions having the second optical state on the substrate, wherein the two sets of local regions are distinguishable from one another by size of local regions, visible as differently sized dark areas bleached in a fluorescent layer. There is a clear difference in size between the sets of local regions, one set of local regions being visible as big dark areas and the other set of local regions being visible as small dark areas. The white areas are artefacts and should be ignored.

Further, the method of the present invention uses two or more reporter reagents to detect two or more analytes. The two or more reporter reagents are different types of reporter reagents to form two or more sets of local regions having the second optical state on the substrate which are distinguishable from one another by rate of formation of the sets of local regions, order of formation of the sets of local regions, or size of local regions optionally in combination with rate of formation of the sets of local regions or order of formation of the sets of local regions.

Additionally, excess particles can be suspended in the sample that are not bound to the surface. If the sample is whole blood, then red cells will be suspended in the medium. Preferably, the substrate 14 forms the top of the sample chamber 13, allowing red blood cells to sediment away from the substrate 14.

A proportion of each of the two or more reporter reagents binds to the surface of the substrate 14 by means of the corresponding binding component. The sample therefore contains bound reporter reagent and unbound reporter reagent free in solution. The depth of the sample chamber 13 is designed to minimise the diffusion path length of the two or more reporter reagents, and allow the two or more reporter reagents to bind rapidly. Typically, the depth of the sample chamber is from 50 to 200 μm.

In the method of the present invention, the sample chamber 13 is filled with a sample containing two or more analytes. Two or more reporter reagents such as photosensitiser-labelled antibodies 3 and 4 are also added to the sample chamber 13. If a whole blood sample is used, the sample may also contain additional components such as red blood cells.

After a period of time, a fraction of each of the two or more reporter reagents such as photosensitiser-labelled antibodies 3 and 4 are bound to the surface of the substrate 14 in proportion to the concentration of the corresponding analyte such as protein analytes 6a and 6b by means of the corresponding binding component such as antibodies 2a and 2b. An excess of reporter reagents is included so that a significant proportion of each of the two or more analytes forms a sandwich complex. A proportion of each of the two or more reporter reagents thus binds to the surface of the substrate 14 in proportion to the concentration of the corresponding analyte, by means of the corresponding binding component. The sample therefore contains bound reporter reagent and unbound reporter reagent free in solution.

After irradiation, the excited photosensitiser interacts with the optical component to cause the optical component to change from a first optical state to a second optical state. For example, streptavidin labelled with optical component in fluorescent state 10 becomes streptavidin labelled with optical component in non-fluorescent state 11. Only the optical component that is in close proximity to the excited photosensitiser changes from the first optical state to the second optical state.

The photosensitiser is proximal to the substrate when the binding event has occurred. That is, the photosensitiser is sufficiently close to the surface of the substrate to interact with the optical component and convert it from the first optical state to the second optical state on irradiation of the device. The actual distance between the photosensitiser and the surface of the substrate will, however, depend on a number of variables, such as the size and nature of the photosensitiser, the size and nature of the binding components, the reporter reagents and the analytes, and the nature of the sample medium.

Any excited photosensitiser in the vicinity of the optical component interacts to cause the optical component to change from the first optical state to the second optical state. In this way, the excited photosensitiser of each of the two or more reporter reagents bound to the surface of the substrate interacts with the optical component to cause the optical component to change from the first optical state to the second optical state, thereby forming two or more sets of local regions having the second optical state on the substrate 14.

The reporter reagent comprising the excited photosensitiser must be permanently bound to the surface of the substrate 14 during the entire period of irradiation in order to achieve complete conversion of the first optical state to the second optical state. If the reporter reagent comprising the excited photosensitiser is only transiently bound to the surface for a fraction of the irradiation period, then there is incomplete conversion to the second optical state, which can be detected by algorithms used to measure the size, shape and intensity of the discrete areas. Any unbound reporter reagent in solution does not cause the optical component to change significantly from the first optical state to the second optical state.

This provides a significant advantage over other digital assay methods in that it removes the need for washing steps. In this way, the method of the present invention is a homogeneous assay. In a conventional assay, the unbound reporter reagent must be separated from the bound reporter reagent before any measurement is taken since the unbound reporter reagent interferes with the signal generated by the bound reporter reagent. However, on account of the localised surface changes provided by the present invention, bound and unbound reporter reagent may be distinguished. Indeed, the ability to distinguish between reporter reagents proximal to the surface of the substrate 14 (i.e. bound) and reporter reagents in the bulk solution (i.e. unbound) is a particular advantage of the present invention. Preferably, steps (i) to (iii) take place in the absence of wash steps, i.e. the method is carried out without removing the sample from the substrate in steps (i), (ii) and (iii).

Step (iv) of the method of the present invention involves optionally detecting the at least one set of local regions of the optical component having the second optical state on the substrate formed in step (iii) and repeating step (iii) with electromagnetic radiation at the same or a different wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from the first optical state to the second optical state, thereby forming at least one set of local regions of the optical component having the second optical state on the substrate.

Step (iv) is optional and occurs when the two or more sets of local regions having the second optical state on the substrate form sequentially in two or more irradiation steps and detection after each irradiation step is required.

If step (iv) is performed, detecting the at least one set of local regions of the optical component having the second optical state on the substrate formed in step (iii) is preferably carried out by image analysis software that can distinguish actual binding events from transient binding events and surface artefacts by analysis of surface intensity and morphology. Detection may be a separate step that is carried out after the optical component has been converted from the first optical state to the second optical state. Alternatively, detection may be carried out during the course of the conversion to the second optical state.

Any method that can successfully identify individual binding events can be used. For example, an initial image of the surface could be taken using the excitation wavelength of the optical component first, followed by irradiation with electromagnetic radiation to excite the photosensitisers, followed by a final step of irradiation with the excitation wavelength of the optical component.

Alternatively, it may be possible to capture a series of images (or video file) of the surface whilst irradiating with electromagnetic radiation that excites both the optical component and the photosensitiser simultaneously. Any combination of optical filters, dichroic mirrors and illumination sequences that allow the binding events to be identified are acceptable.

The second optical state forms a set of local regions on the substrate. Advantageously, local regions having the second optical state can be counted as individual binding events. The method of the present invention is thus suitable for performing a digital assay. However, if there are a large number of binding events such that the majority of the optical component is in the second optical state, the bulk change may be detected.

In a preferred embodiment, the at least one set of local regions having the second optical state on the substrate is detected by counting local regions in each set of local regions having the second optical state on the substrate or by measuring the at least one set of local regions having the second optical state on the substrate as a bulk property. More preferably, the at least one set of local regions having the second optical state on the substrate is detected by counting local regions in each set of local regions having the second optical state on the substrate.

Local regions having the second optical state may need to be above or below a threshold value corresponding to a background signal, depending on the first and second optical states. For example, when the first optical state is non-fluorescent and the second optical state is fluorescent, the level of fluorescence of the second optical state may need to be above a threshold value before being detected. This would eliminate any interference from autofluorescence of the sample. Alternatively, when the first optical state is fluorescent and the second optical state is non-fluorescent, the level of fluorescence of the second optical state may need to be below a threshold value before being detected.

The local regions having the second optical state on the substrate are typically discrete areas on the substrate. However, some local regions may be excluded from detection owing to their morphology. Local regions corresponding to an individual binding event tend to be uniform and circular but some local regions may be irregular in shape, corresponding to artefacts. Further, some local regions may be larger than others where particles have clumped together, or they may be smaller, where there has only been a transient binding event. Therefore, in a preferred embodiment, only uniform, circular local regions having the second optical state on the substrate are detected.

The at least one set of local regions on the substrate can be detected using simple optical means. In a preferred embodiment, the at least one set of local regions having the second optical state on the substrate is detected using optical microscopy. An optical set-up suitable for detection is shown in FIG. 6. More preferably, the at least one set of local regions having the second optical state on the substrate is detected using wide-field microscopy. Wide-field microscopy is the simplest form of microscopy, whereby the whole sample is illuminated and imaged at the same time, in comparison to more complex techniques such as confocal microscopy where only one single focal spot is illuminated and recorded at a time. The benefits of confocal microscopy are increased contrast by removal of out-of-focus haze and the ability to take a stack of images through the depth of the sample. Super-resolution microscopy techniques are also known, such as photo-activated localization microscopy (PALM or FPALM) and stochastic optical reconstruction microscopy (STORM). These methods have added complexity and cost over simple wide-field methods.

In order to detect the at least one set of local regions on the substrate, a wide-field fluorescent microscope with a light source (for example, an LED) and a photodetector (for example a camera, such as a CCD) can be used, using excitation and emission filters suitable for the detection of the particular optical component.

If step (iv) is performed, step (iii) is repeated in the same way as irradiation step (iii), except with electromagnetic radiation at the same or a different wavelength. The preferred embodiments of step (iii) also apply to irradiating in step (iv).

In a preferred embodiment, optional step (iv) of the method of the present invention involves repeating step (iii) with electromagnetic radiation at the same wavelength. This provides a simpler method for detecting and distinguishing between two or more analytes in a sample than typical methods for measuring two or more analytes in a sample in a homogeneous assay format which use electromagnetic radiation at different wavelengths. This method may be used when the two or more reporter reagents differ from one another by size, gas permeability, reactivity of the photosensitiser, amount of the photosensitiser or combinations thereof. In this case, the electromagnetic radiation used in step (iii) and optional step (iv) corresponds to the excitation wavelength of the photosensitiser of the two or more reporter reagents. In this embodiment, the two or more sets of local regions are distinguishable from one another by rate of formation of the sets of local regions, or size of local regions in combination with rate of formation of the sets of local regions.

Preferably, optional step (iv) of the method of the present invention involves repeating step (iii) with electromagnetic radiation at the same wavelength for a longer period of time and/or at a higher intensity. This allows the set of local regions that forms at a slower rate to then form.

In an alternative preferred embodiment, optional step (iv) of the method of the present invention involves repeating step (iii) with electromagnetic radiation at a different wavelength. This method is used when the two or more reporter reagents differ from one another by excitation wavelength of the photosensitiser optionally in combination with size. In this case, the electromagnetic radiation used in step (iii) and optional step (iv) corresponds to the excitation wavelengths of the photosensitisers of the two or more reporter reagents. In this embodiment, the two or more sets of local regions are distinguishable from one another by order of formation of the sets of local regions, or size of local regions in combination with order of formation of the sets of local regions.

Step (v) of the method of the present invention involves if step (iv) is performed, detecting the at least one set of local regions of the optical component having the second optical state on the substrate formed in step (iv) or, if step (iv) is not performed, detecting two or more sets of local regions having the second optical state on the substrate formed in step (iii), wherein the two or more sets of local regions are distinguishable from one another by rate of formation of the sets of local regions, order of formation of the sets of local regions, or size of local regions optionally in combination with rate of formation of the sets of local regions or order of formation of the sets of local regions.

Therefore, if step (iv) is not performed, step (iii) is irradiating the device with electromagnetic radiation at one wavelength for absorption by the photosensitiser of each of the two or more reporter reagents, such that the photosensitiser of each of the two or more reporter reagents bound to the surface of the substrate absorbs electromagnetic radiation and interacts with the optical component to cause the optical component to change from the first optical state to the second optical state, thereby forming two or more sets of local regions of the optical component having the second optical state on the substrate. In this instance, the sets of local regions differ by size.

Step (v) is performed in the same way as detecting in optional step (iv). The preferred embodiments of detecting in optional step (iv) also apply to step (v).

In the method of the present invention, two or more analytes in a sample are detected resulting in the formation of two or more sets of local regions having the second optical state on the substrate. The two or more sets of local regions are distinguishable from one another by rate of formation of the sets of local regions, order of formation of the sets of local regions, or size of local regions optionally in combination with rate of formation of the sets of local regions or order of formation of the sets of local regions.

That is, the two sets of local regions are distinguishable from one another by rate of formation of the sets of local regions, order of formation of the sets of local regions or size of local regions. And if the two sets of local regions are distinguishable from one another by size of local regions, the two sets of local regions are optionally additionally distinguishable from one another by rate of formation of the sets of local regions or order of formation of the sets of local regions. That is, the two sets of local regions may be distinguishable from one another by size of local regions and rate of formation of the sets of local regions. Or the two sets of local regions may be distinguishable from one another by size of local regions and order of formation of the sets of local regions.

The difference between rate of formation and order of formation is that when the two sets of local regions are distinguishable from one another by rate of formation, the two sets of local regions will always form in the same order when using excitation at the same wavelength whereas when the two sets of local regions are distinguishable from one another by order of formation, the order of formation of the two sets of local regions can be switched by changing the order in which the two excitation wavelengths are used.

By rate of formation of the sets of local regions is meant the length of time for a set of local regions to form to a given size using electromagnetic radiation at one wavelength.

In a preferred embodiment, at least two of the two or more sets of local regions are distinguishable from one another by rate of formation of the sets of local regions. Preferably, the two or more sets of local regions are distinguishable from one another by rate of formation of the sets of local regions. This is an obvious difference and the concentration for one analyte can be calculated by counting the number of local regions in the set of local regions after a first irradiation step and the concentration for another analyte can be calculated by counting the number of local regions in the set of local regions after a second irradiation step using the same wavelength. This provides a simple method for detecting and distinguishing between two or more analytes in a sample.

In contrast to typical methods for measuring two or more analytes in a sample in a homogeneous assay format, there is therefore no requirement in the method of the present invention to use electromagnetic radiation at different wavelengths to detect two or more analytes in a sample. This further simplifies detecting and distinguishing between two or more analytes in a sample.

By order of formation of the sets of local regions is meant that the sets of local regions form sequentially in response to irradiating the device with different excitation wavelengths of the photosensitisers.

In a preferred embodiment, at least two of the two or more sets of local regions are distinguishable from one another by order of formation of the sets of local regions. Preferably, the two or more sets of local regions are distinguishable from one another by order of formation of the sets of local regions. Again, this is an obvious difference and the concentration for one analyte can be calculated by counting the number of local regions in the set of local regions formed after a first irradiation step and the concentration for another analyte can be calculated by counting the total number of local regions in the sets of local regions formed after a second irradiation step using a different wavelength and subtracting the number of local regions in the set of local regions formed after the first irradiation step. This provides a simple method for detecting and distinguishing between two or more analytes in a sample.

By size of local regions is meant size of local regions at a given time or maximum size attainable, preferably maximum size attainable.

The size of each local region in a set of local regions of the optical component having the second optical state on the substrate should be the same and the local regions in one set of local regions should be clearly bigger or smaller than the local regions in another set of local regions. Preferably, the size of each local region in a set of local regions of the optical component having the second optical state on the substrate is the same.

As discussed above, local regions corresponding to an individual binding event tend to be uniform and circular. Therefore, the two or more sets of local regions are preferably distinguishable from one another by diameter of local regions.

In a preferred embodiment, at least two of the two or more sets of local regions are distinguishable from one another by size of local regions. Preferably, the two or more sets of local regions are distinguishable from one another by size of local regions. Again, this is an obvious difference and the concentrations of the two or more analytes can simply be calculated by counting the number of local regions in each set of local regions. This provides a simple method for detecting and distinguishing between two or more analytes in a sample.

In another preferred embodiment, the two or more sets of local regions are preferably distinguishable from one another by size of local regions and rate of formation of the sets of local regions.

Alternatively, the two or more sets of local regions are preferably distinguishable from one another by size of local regions and order of formation of the sets of local regions.

The use of two or more different reporter reagents results in the two or more sets of local regions being distinguishable from one another by rate of formation of the sets of local regions, order of formation of the sets of local regions, or size of local regions optionally in combination with rate of formation of the sets of local regions or order of formation of the sets of local regions.

The two or more reporter reagents have different physicochemical properties. There are a number of physicochemical properties that may be varied so that the two or more sets of local regions are distinguishable from one another by rate of formation of the sets of local regions, order of formation of the sets of local regions, or size of local regions optionally in combination with rate of formation of the sets of local regions or order of formation of the sets of local regions. These physicochemical properties include size, material properties (e.g. gas permeability), type of photosensitiser (e.g. wavelength at which it can be activated, reactivity of photosensitiser) and/or amount of photosensitiser.

In a preferred embodiment, the two or more reporter reagents differ from one another by size, gas permeability, excitation wavelength of the photosensitiser, reactivity of the photosensitiser, amount of the photosensitiser or combinations thereof. More preferably, the two or more reporter reagents differ from one another by size, gas permeability, excitation wavelength of the photosensitiser, amount of the photosensitiser or combinations thereof. Most preferably, the two or more reporter reagents differ from one another by size and/or excitation wavelength of the photosensitiser.

Preferably, at least two of the two or more reporter reagents differ from one another by size. If the effect of the photosensitiser on the optical component is limited to a finite distance from the reporter reagent, then a larger reporter reagent can interact with the optical component at a greater distance from the centre of the reporter reagent. Additionally, a greater size allows a larger amount of photosensitiser to be associated with the reporter reagent, increasing the activity of the reporter reagent. This increased activity may facilitate a faster interaction between the photosensitiser of the reporter reagent and the optical component, leading to more rapid formation of a set of local regions, which also are greater in size. As such, at least two of the two or more sets of local regions are preferably distinguishable from one another by size of local regions optionally in combination with rate of formation of the sets of local regions. Preferably, the two or more reporter reagents differ from one another by size.

Preferably, at least two of the two or more reporter reagents differ from one another by gas permeability. For example, greater gas permeability facilitates a faster interaction between the photosensitiser of the reporter reagent and the optical component, leading to more rapid formation of a set of local regions and/or a set of local regions having local regions of greater size. As such, at least two of the two or more sets of local regions are distinguishable from one another by rate of formation of the sets of local regions or size of local regions optionally in combination with rate of formation of the sets of local regions. Preferably, the two or more reporter reagents differ from one another by gas permeability

Preferably, at least two of the two or more reporter reagents differ from one another by excitation wavelength of the photosensitiser. As such, at least two of the two or more sets of local regions are distinguishable from one another by order of formation of the sets of local regions. Preferably, the two or more reporter reagents differ from one another by excitation wavelength of the photosensitiser.

This can be seen in FIGS. 7-11. FIG. 7 shows a device in which two reporter reagents comprising photosensitisers which are stimulated by different wavelengths of radiation are bound to the surface of the substrate before irradiation. Photosensitiser-labelled antibody 3 is bound to the surface of the substrate by means of antibody 2a and photosensitiser-labelled antibody 5 is bound to the surface of the substrate by means of antibody 2b. Photosensitiser-labelled antibodies 3 and 5 act as the reporter reagents.

FIG. 8 shows the device of FIG. 7 being irradiated with electromagnetic radiation at one wavelength, for example 680 nm. The light source may be, for example, LED 15a. The light source illuminates the sample chamber 13 with light of the appropriate wavelength to excite the photosensitiser 1a of photosensitiser-labelled antibody 3.

FIG. 9 shows the device of FIG. 8 after irradiation. The photosensitiser 1a of photosensitiser-labelled antibody 3 interacts with the dye optical component of the streptavidin-dye conjugate 10 to cause the dye to change from a fluorescent state to a non-fluorescent state. Streptavidin-dye conjugate in fluorescent state 10 becomes streptavidin-dye conjugate in non-fluorescent state 11. Only the dye that is in close proximity to photosensitiser 1a changes from the first optical state to the second optical state to form a set of local regions of the optical component having the second optical state on the substrate.

This set of local regions can then be detected before irradiating the device with electromagnetic radiation at a different wavelength for absorption by photosensitiser 1b of photosensitiser-labelled antibody 5.

FIG. 10 shows the device of FIG. 9 being irradiated with electromagnetic radiation at a different wavelength, for example 350 nm. The light source may be, for example, LED 15b. The light source illuminates the sample chamber 13 with light of the appropriate wavelength to excite the photosensitiser 1b of photosensitiser-labelled antibody 5.

FIG. 11 shows the device of FIG. 10 after irradiation. The photosensitiser 1b of photosensitiser-labelled antibody 5 interacts with the dye optical component of the streptavidin-dye conjugate 10 to cause the dye to change from a fluorescent state to a non-fluorescent state. Streptavidin-dye conjugate in fluorescent state 10 becomes streptavidin-dye conjugate in non-fluorescent state 11. Only the dye that is in close proximity to photosensitiser 1b changes from the first optical state to the second optical state to form an additional set of local regions of the optical component having the second optical state on the substrate.

This additional set of local regions can then be detected. As such, the two sets of local regions are distinguishable from one another by order of formation of the sets of local regions.

Preferably, at least two of the two or more reporter reagents differ from one another by reactivity of the photosensitiser. For example, the photosensitiser of one reporter reagent may absorb more electromagnetic radiation and/or interact with the optical component to a greater extent than the photosensitiser of another reporter reagent, leading to more rapid formation of a set of local regions and/or a set of local regions having local regions of greater size. As such, at least two of the two or more sets of local regions are distinguishable from one another by rate of formation of the sets of local regions or size of local regions optionally in combination with rate of formation of the sets of local regions. Preferably, the two or more reporter reagents differ from one another by reactivity of the photosensitiser.

Preferably, at least two of the two or more reporter reagents differ from one another by amount of the photosensitiser. For example, a reporter reagent containing a higher amount of photosensitiser will absorb more electromagnetic radiation and interact with the optical component to a greater extent than another reporter reagent containing less photosensitiser, leading to more rapid formation of a set of local regions and/or a set of local regions having local regions of greater size. As such, at least two of the two or more sets of local regions are distinguishable from one another by rate of formation of the sets of local regions or size of local regions optionally in combination with rate of formation of the sets of local regions. Preferably, the two or more reporter reagents differ from one another by amount of the photosensitiser.

The two or more reporter reagents may differ from one another by one or more than one physicochemical property. For example, four analytes in a sample could be detected using two reporter reagents which differ from one another by size and two reporter reagents which differ from one another by excitation wavelength of the photosensitiser, wherein the four sets of local regions formed are distinguishable from one another by size of local regions and order of formation of the sets of local regions.

In a preferred embodiment, the method of the present invention comprises the steps of:
(i) providing a mixture comprising a sample and two or more reporter reagents to a device, wherein each of the two or more reporter reagents comprises a photosensitiser, the device comprising a substrate having an optical component and two or more binding components, wherein the optical component and the two or more binding components are attached to a surface of the substrate;
(ii) allowing a portion of each of the two or more reporter reagents to bind to the surface of the substrate in proportion to the concentration of the corresponding analyte, by means of the corresponding binding component;
(iii) irradiating the device with electromagnetic radiation at one wavelength for absorption by the photosensitiser of each of the two or more reporter reagents, such that the photosensitiser of each of the two or more reporter reagents bound to the surface of the substrate absorbs electromagnetic radiation and interacts with the optical component to cause the optical component to change from the first optical state to the second optical state, thereby forming two or more sets of local regions of the optical component having the second optical state on the substrate; and
(iv) detecting the two or more sets of local regions having the second optical state on the substrate,
wherein the two or more sets of local regions are distinguishable from one another by size of local regions.

In this embodiment, the two or more reporter reagents preferably differ from one another by size, gas permeability, reactivity of the photosensitiser, amount of the photosensitiser or combinations thereof.

In another preferred embodiment, the method of the present invention comprises the steps of:
(i) providing a mixture comprising a sample and two or more reporter reagents to a device, wherein each of the two or more reporter reagents comprises a photosensitiser, the device comprising a substrate having an optical component and two or more binding components, wherein the optical component and the two or more binding components are attached to a surface of the substrate;
(ii) allowing a portion of each of the two or more reporter reagents to bind to the surface of the substrate in proportion to the concentration of the corresponding analyte, by means of the corresponding binding component;
(iii) irradiating the device with electromagnetic radiation at one wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) detecting the at least one set of local regions of the optical component having the second optical state on the substrate formed in step (iii) and repeating step (iii) with electromagnetic radiation at the same or a different wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from the first optical state to the second optical state, thereby forming at least one set of local regions of the optical component having the second optical state on the substrate; and
(v) detecting the at least one set of local regions of the optical component having the second optical state on the substrate formed in step (iv),
wherein the two or more sets of local regions are distinguishable from one another by rate of formation of the sets of local regions, order of formation of the sets of local regions, or size of local regions in combination with rate of formation of the sets of local regions or order of formation of the sets of local regions.

In this embodiment, the two or more reporter reagents preferably differ from one another by size, gas permeability, excitation wavelength of the photosensitiser, reactivity of the photosensitiser, amount of the photosensitiser or combinations thereof.

In a preferred embodiment, the method of the present invention comprises the steps of:
(i) providing a mixture comprising a sample and two or more reporter reagents to a device, wherein each of the two or more reporter reagents comprises a photosensitiser, the device comprising a substrate having an optical component and two or more binding components, wherein the optical component and the two or more binding components are attached to a surface of the substrate;
(ii) allowing a portion of each of the two or more reporter reagents to bind to the surface of the substrate in proportion to the concentration of the corresponding analyte, by means of the corresponding binding component;
(iii) irradiating the device with electromagnetic radiation at one wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) detecting the at least one set of local regions of the optical component having the second optical state on the substrate formed in step (iii) and repeating step (iii) with electromagnetic radiation at the same wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from the first optical state to the second optical state, thereby forming at least one set of local regions of the optical component having the second optical state on the substrate; and
(v) detecting the at least one set of local regions of the optical component having the second optical state on the substrate formed in step (iv),
wherein the two or more sets of local regions are distinguishable from one another by rate of formation of the sets of local regions or size of local regions in combination with rate of formation of the sets of local regions.

In this embodiment, the two or more reporter reagents preferably differ from one another by size, gas permeability, reactivity of the photosensitiser, amount of the photosensitiser or combinations thereof.

In another preferred embodiment, the method of the present invention comprises the steps of:
(i) providing a mixture comprising a sample and two or more reporter reagents to a device, wherein each of the two or more reporter reagents comprises a photosensitiser, the device comprising a substrate having an optical component and two or more binding components, wherein the optical component and the two or more binding components are attached to a surface of the substrate;
(ii) allowing a portion of each of the two or more reporter reagents to bind to the surface of the substrate in proportion to the concentration of the corresponding analyte, by means of the corresponding binding component;
(iii) irradiating the device with electromagnetic radiation at one wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) detecting the at least one set of local regions of the optical component having the second optical state on the substrate formed in step (iii) and repeating step (iii) with electromagnetic radiation at a different wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from the first optical state to the second optical state, thereby forming at least one set of local regions of the optical component having the second optical state on the substrate; and
(v) detecting the at least one set of local regions of the optical component having the second optical state on the substrate formed in step (iv),
wherein the two or more sets of local regions are distinguishable from one another by order of formation of the sets of local regions or size of local regions in combination with order of formation of the sets of local regions.

In this embodiment, the two or more reporter reagents preferably differ from one another by excitation wavelength of the photosensitiser optionally in combination with size.

In a preferred embodiment, the two or more binding components are attached to the same area on the surface of the substrate. The two or more analytes are distinguished by rate of formation of the sets of local regions, order of formation of the sets of local regions, or size of local regions optionally in combination with rate of formation of the sets of local regions or order of formation of the sets of local regions. In contrast to typical methods for measuring two or more analytes in a sample in a homogeneous assay format, there is therefore no requirement to separate the two or more binding components on the surface of the substrate. A smaller sample volume is also required for a multiplexed assay as the method of the present invention uses a single substrate. This provides a simple method for detecting and distinguishing between two or more analytes in a sample.

In an alternative preferred embodiment, each of the two or more binding components is attached to a separate area of the surface of the substrate. Each of these separate areas contains only one specific type of binding component. Therefore, only one type of reporter reagent should bind to each separate area in the presence of the corresponding analyte thus promoting binding of specific reporter reagent to specific binding component. If these areas were interrogated separately, then, in principle, there is no requirement to distinguish between sets of local regions. The specific analyte concentrations could just be calculated from the sets of local regions formed in the separate areas. However, the fact that there are distinguishable sets of local regions means that specific binding of the reporter reagent expected to bind and non-specific binding of unwanted reporter reagent can be distinguished. Non-specific binding of reporter reagent is common to all immunoassays and is often compensated for by carrying out a control measurement to monitor the level of non-specific binding, as described in further detail below. In this particular embodiment, the separate areas can act as negative controls for one another as shown in Examples 3 and 4. These negative controls can compensate for variability between different samples. This reduced interference and increased accuracy is a significant advantage over multiplexed assays that add all the different reporter reagents to one sample and then distinguish binding solely by varying the binding component by location.

The photosensitiser of each of two or more reporter reagents bound to the surface of the substrate may interact directly with the optical component to cause the change (e.g. the photosensitiser is excited and transfers this energy directly to the optical component) or indirectly with the optical component to cause the change via an additional reagent (e.g. the photosensitiser is excited and transfers this energy to an additional component, which then transfers this energy to the optical component).

In a preferred embodiment, the photosensitiser of each of the two or more reporter reagents absorbs electromagnetic radiation and interacts with a pre-activator reagent present in the mixture to generate an activator reagent which interacts with the optical component to cause the optical component to change from the first optical state to the second optical state. Therefore, the two or more reporter reagents are capable of generating an activator reagent from a pre-activator reagent present in the mixture upon absorption of electromagnetic radiation, and the optical component is capable of changing from a first optical state to a second optical state on interaction with the activator reagent.

The pre-activator reagent may be present in the sample or the pre-activator reagent may be added to the mixture of the sample and the two or more reporter reagents as an additional reagent. In a preferred embodiment, the pre-activator reagent is ground state triplet oxygen. In another preferred embodiment, the activator reagent is a reactive oxygen species (ROS). Preferably, the ROS is selected from a hydroxyl radical, superoxide, peroxide, organic peroxides, peroxynitrite, singlet oxygen and mixtures thereof. More preferably, the ROS is singlet oxygen. Singlet oxygen is the preferred activator reagent because it has a short half-life and finite diffusion path length (normally less than 1 micron under aqueous conditions).

It is believed that the photosensitiser absorbs the light to generate an excited state which can undergo intersystem crossing (ISC) with oxygen present in the sample and proximal to the reporter reagent to generate singlet oxygen. Singlet oxygen then goes on to interact with the optical component as discussed below. In a particularly preferred embodiment, the pre-activator reagent is ground state triplet oxygen and the activator reagent is singlet oxygen.

Singlet oxygen has previously been used in immunoassays such as the luminescent oxygen channelling immunoassay (LOCI). The LOCI immunoassay is a homogenous non-digital assay using donor and acceptor beads that measures a bulk phenomenon. The donor beads generate singlet oxygen upon illumination at 680 nm, and the acceptor beads generate a chemiluminescent signal when activated by singlet oxygen. Binding of donor to acceptor beads is promoted by antibody-antigen binding. The reaction mixture is typically illuminated for 0.5 to 1.0 seconds and then the luminescence signal is measured for 0.5 to 1.0 seconds. Critically, the measurement takes place in the presence of all unbound donor and acceptor beads. Spatial separation of the beads minimises background signal; however, the LOCI assay cannot distinguish between long-lived and transient binding events, due to the short measurement time. The assay can achieve a detection limit of around 1-5 μg/mL for its most sensitive assays, for example interleukin 6 (IL-6) or thyroid stimulating hormone (TSH). The method of the present invention minimises background signal even further because the “donor” particles, the reporter reagent, needs to be in proximity to the surface of the substrate 11, rather than to particles in solution, in order for a signal to be detected, and must also be there for the duration of the illumination period in order to generate a signal that is above (or below) threshold criteria used in the image analysis software. Therefore, the method of the present invention is more sensitive and can detect analytes in lower concentrations than the LOCI assay.

In a preferred embodiment, the optical component is a dye. Preferably, the optical component is selected from one of the following fluorescent dyes and mixtures thereof:

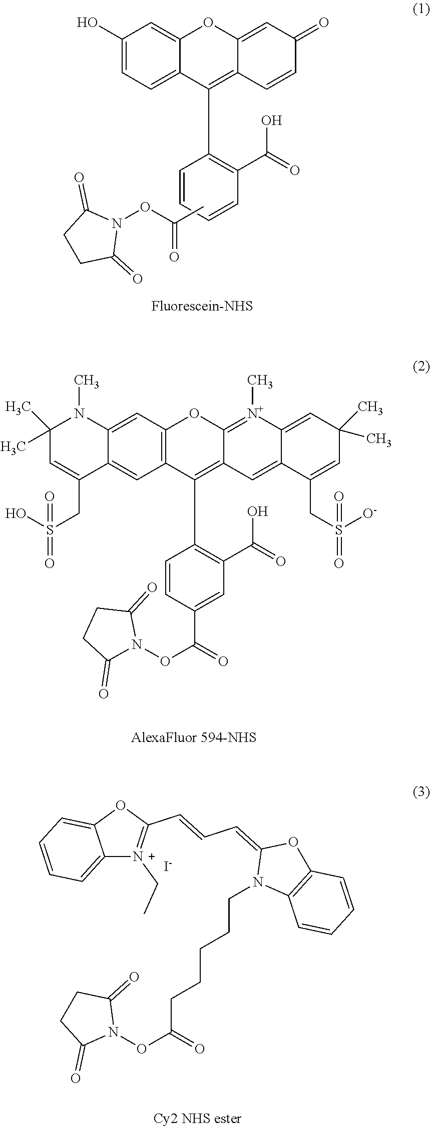

Dyes (1), (2) and (3) are common organic fluorophores that can be attached to proteins and other macromolecules by means of the attached N-hydroxy succinimide groups, which react with amine groups to form a covalent amide bond. These dyes, along with a wide range of other fluorophores can be irreversibly converted into a non-fluorescent state by the method of the present invention.

Preferably, the optical component when in one of the first and second optical states is fluorescent and the optical component when in the other of the first and second optical states is non-fluorescent. In one embodiment, the optical component in the first optical state is fluorescent and in the second optical state is non-fluorescent or the optical component in the first state is non-fluorescent and in the second optical state is fluorescent. More preferably however, the optical component when in the first optical state is fluorescent and the optical component when in the second optical state is non-fluorescent.

Preferably, the change from the first optical state to the second optical state is irreversible. This allows for subsequent scanning of the substrate to identify areas in which the change in optical state has taken place.

The assay requires the presence of two or more binding components, the number of binding components corresponding to the number of analytes being detected.

The two or more binding components have a binding site which is capable of binding a corresponding reporter reagent proportionally to the concentration of the corresponding analyte in the sample. The proportionality is important for the functioning of the assay since the binding must be dependent on the concentration of the analyte for any meaningful measure of the concentration of the analyte to be determined. The binding may be directly proportional or indirectly proportional to the concentration of the analyte depending on the type of assay being performed. In the case of a non-competitive assay, e.g. an immunometric assay, the binding is directly proportional to the concentration of the analyte, but for a competitive assay, the binding is indirectly proportional to the concentration of the analyte.

One particular type of competitive assay is presented in which an antibody to the analyte is immobilised on the substrate, and a labelled analogue of the analyte is introduced into the sample. The analyte and labelled analogue of the analyte then “compete” for the antibody on the surface. In the absence of analyte, then the labelled analogue will bind at the maximum possible rate. However, in the presence of analyte, the antibody on the substrate becomes populated with analyte and the rate of binding of the analogue is diminished.

The two or more binding components may be adapted to bind to the corresponding analyte, or a complex or derivative of the corresponding analyte, in which case the two or more reporter reagents will bind to the corresponding binding component in the presence of the corresponding analyte, or the complex or derivative of the corresponding analyte. In this case, the two or more binding components have a binding site which is capable of binding to the corresponding reporter reagent in the presence of the corresponding analyte or the complex or derivative of the corresponding analyte. The binding is, however, still proportional to the concentration of the corresponding analyte.

Alternatively, the two or more binding components may themselves be an analogue of the corresponding analyte and the corresponding reporter reagent binds directly to the two or more binding components (it is an analogue because it is bound to the surface of the substrate either through covalent bonding or non-covalent interactions). In this case, the two or more binding components will compete with the unbound analyte, or an unbound complex or derivative of the analyte, for the binding of the corresponding reporter reagent. Accordingly, the two or more binding components will simply be capable of binding to the corresponding reporter reagent.

Determining the extent of binding of the two or more reporter reagents to the corresponding binding component (either directly or mediated by the corresponding analyte/complex or derivative of the corresponding analyte) provides a measurement of the concentration of the corresponding analyte in the sample. It is customary that the system has been pre-calibrated at the point of manufacture to generate a calibration curve that is used to convert the instrument signals into the measured analyte concentrations in the sample.

The assay also requires the presence of two or more reporter reagents, the number of reporter reagents corresponding to the number of analytes being detected.

Each of the two or more reporter reagents comprises a photosensitiser. The photosensitiser is capable of absorbing electromagnetic radiation to interact with the optical component. It is this interaction which causes the optical component to change from the first optical state to the second optical state.

The photosensitiser may therefore be composed of any material which is capable of interacting with the electromagnetic radiation in this manner. Suitable photosensitisers are known from photodynamic therapy (PDT) as PDT reagents. PDT reagents are used in cancer therapy and dermatology to destroy cells upon illumination (Shafirstein et al., Cancers, 2017, 9, 12; Wan and Lin, Clinical, Cosmetic and Investigational Dermatology, 2014, 7, 145).

Upon irradiation with electromagnetic radiation, PDT reagents are promoted to an excited triplet state. This excited triplet state can interact directly with cellular components, in what is called a Type I process or can interact with oxygen in what is called a Type II process. Both Type I and Type II processes can lead to the formation of ROS. In the Type II process, the predominant product is singlet oxygen, through an intersystem crossing mechanism.

Singlet oxygen is an excited state of oxygen that is highly reactive. It can undergo a number of reactions before decaying, including Diels-Alder type reactions and ene reactions. It also undergoes general oxidation reactions with sulfur and nitrogen containing compounds. The indiscriminate reactivity of singlet oxygen is one of the reasons why it is used in photodynamic therapy.

A wide range of photosensitiser compounds are known, including porphyrins, chlorins (for example pyropheophorbide-a), phthalocyanines and other polyaromatic species (see, for example, Antibody-Directed Phototherapy, Pye et al., Antibodies, 2013, 2, 270).

In one embodiment, the photosensitiser is selected from porphyrins, chlorins, phthalocyanines and other polyaromatic species. In a preferred embodiment, the photosensitiser is a silicon phthalocyanine derivative, such as the example shown below:

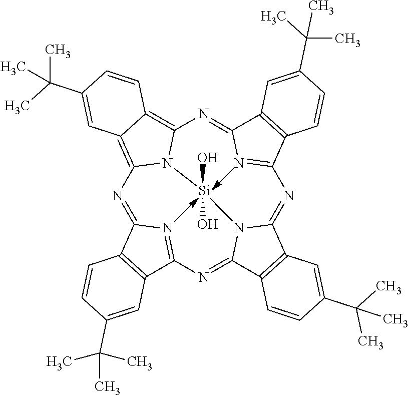

The nature of the binding components and the reporter reagents will depend on the nature of the analyte, but they preferably comprise antibodies. The method of the present invention has particular applicability in immunoassays. In a particularly preferred embodiment, the two or more binding components are antibodies raised to the corresponding analyte or the complex or derivative of the corresponding analyte, and the two or more reporter reagents comprise antibodies raised to the corresponding analyte or the complex or derivative of the corresponding analyte. In principle, a single molecule could be used for each reagent, but in practice, the binding components and the reporter reagents are a population of molecules. The term “antibody” preferably includes within its scope a Fab fragment, a single-chain variable fragment (scFv), and a recombinant binding fragment.

As alternatives to antibody-antigen reactions, the binding components, the reporter reagents and the analytes may be a first and second nucleic acid where the first and second nucleic acids are complementary, or reagents containing avidin or derivatives thereof and analytes containing biotin or derivatives thereof, or vice versa. The binding components and the reporter reagents may also be aptamers. The system is also not limited to biological assays and may be applied, for example, to the detection of heavy metals in water. The system also need not be limited to liquids and any fluid system may be used, e.g. the detection of enzymes, cells and viruses etc. in the air.

The photosensitiser may be on the inside or the outside of each of the two or more reporter reagents. Preferably, each of the two or more reporter reagents comprises a polymer particle and the photosensitiser is encapsulated within the polymer particle. Alternatively, each of the two or more reporter reagents further comprises a polymer particle and the photosensitiser is coated on the polymer particle surface. Suitable polymer particles include latex particles, generally made from polystyrene or co-polymers of polystyrene. These polymer particles can contain functional groups on the surface, such as carboxyl groups that can be used to form covalent bonds. Such polymer particles swell in non-polar solvents, allowing infusion/encapsulation of hydrophobic organic molecules.

More preferably, each of the two or more reporter reagents further comprises a polymer particle and one or more binding domains, wherein the photosensitiser is encapsulated within the polymer particle and the one or more binding domains is coated on the polymer particle. Alternatively, each of the two or more reporter reagents further comprises a polymer particle and one or more binding domains, wherein the photosensitiser and the one or more binding domains are co-coated on the polymer particle. The binding domain may be an antibody or a nucleic acid etc. as discussed above based on the nature of the analyte. Preferably however, the binding domain is an antibody.

The maximum observable signal is the maximum signal that can be achieved when monitoring the photosensitiser binding to a surface. The binding of particles to the substrate is governed by the diffusion rate of the analytes and reporter reagents which is, in turn, governed largely by the hydrodynamic radius of these components and the viscosity/temperature of the sample.

The device used in the method of the present invention may further comprise controls which compensate for natural variability in the components of the measuring system, variability in the samples that are measured, and variability in the environmental conditions during the measurement. This can be achieved by exposing the sample to reagents on the surface of the substrate. The different reagents are typically located at different areas of the surface of the substrate, these areas being coated in different reagents. These controls are defined as “negative” and “positive” controls, in the sense that the negative control should approximate the expected signal in the absence of analyte, and the positive control should approximate the expected signal when analyte has saturated the system.

To achieve detection with these controls, the device of the present invention preferably comprises two or more binding components with a negative control reagent and a positive control reagent for the two or more binding components, each of which is attached to the surface of the substrate as described above. The negative control reagent and the positive control reagent could be a single negative control reagent and a single positive control reagent for all of the two or more binding components or each of the two or more binding components could have its own negative control reagent and positive control reagent.

However, a negative control reagent may not be required when each of the two or more binding components is attached to a separate area of the surface of the substrate as the separate areas can act as negative controls for each other as described above and in Examples 3 and 4. Therefore, the device of the present invention preferably comprises two or more binding components with a positive control reagent, each of which is attached to the surface of the substrate as described above. The positive control reagent could be a single positive control reagent for all of the two or more binding components or each of the two or more binding components could have its own positive control reagent.

The two or more binding components are as described above.

The negative control reagent has a lower affinity for the two or more reporter reagents under the conditions of the assay than the corresponding binding component. Accordingly, the negative control reagent provides the negative control. It is important that the affinity is considered under the conditions of the assay. The reason is that in the case of a non-competitive assay, the affinity of the two or more binding components for the corresponding reporter reagent is mediated by the presence of the corresponding analyte, or the complex or derivative of the corresponding analyte. Thus, in the absence of the corresponding analyte, or the complex or derivative of the corresponding analyte, neither the two or more binding components nor negative control reagent has any affinity for the corresponding reporter reagent. However, in the presence of the corresponding analyte, or the complex or derivative of the corresponding analyte, the negative control reagent has a lower affinity for the two or more reporter reagents than the corresponding binding component.

In addition, in the embodiments where the two or more binding components bind to the corresponding analyte, or the complex or derivative of the corresponding analyte, the negative control reagent preferably has a lower affinity for the corresponding analyte or, if used, the complex or derivative of the corresponding analyte than the two or more binding components. The negative control reagent is preferably a protein and more preferably an antibody. The negative control reagent typically has similar chemical and physical properties to the two or more binding components, but provides little or no affinity for the corresponding reporter reagent under the conditions of the assay. In a particularly preferred embodiment, the negative control reagent has essentially no affinity for the two or more reporter reagents under the conditions of the assay. Preferably, the negative control reagent provides essentially no affinity for the corresponding analyte or the complex or derivative of the corresponding analyte. That is, the binding of the two or more reporter reagents, or, where applicable, the corresponding analyte or the complex or derivative of the corresponding analyte, to the negative control reagent is non-specific. In this manner, the negative control reagent can compensate for non-specific binding of the two or more reporter reagents to the corresponding binding component. In certain embodiments, a software algorithm uses the data from the negative control region as part of the calculation to give the analyte concentrations.

The positive control reagent binds to the two or more reporter reagents and has an affinity for the two or more reporter reagents which is less influenced by the concentration in the sample of the corresponding analyte or, if used, the complex or derivative of the corresponding analyte than the corresponding binding component and hence provides the positive control. Preferably, the positive control reagent has an affinity for the two or more reporter reagents which is essentially independent of the concentration of the corresponding analyte or the complex or derivative of the corresponding analyte. More preferably, the positive control reagent has a higher affinity for the two or more reporter reagents under the conditions of the assay than the corresponding binding component. In this manner, the positive control reagent measures the maximum expected signal in the system. If the binding of the positive control reagent to the two or more reporter reagents is mediated through a mechanism where the positive control reagent recognises all different types of reporter reagents, it may be possible to measure the binding of all of the two or more reporter reagents in a single positive control area and still be able to distinguish them from each other (i.e. to have a range of positive control measurements).

For both the negative and positive control measurements, a software algorithm may detect unusual binding patterns that could lead to an error message and abort the measurement process.

Although the description above allows the assay to incubate for a finite time before activating the photosensitiser, it is also possible that the kinetics of the binding events could be monitored by irradiating the photosensitiser for discrete periods of time over a time-course and monitoring the binding events as they take place over the course of the reaction.

The present invention is used to detect the presence of multiple analytes in the same sample at the same time. Sandwich and competitive assays may be run in parallel and the assays may use the same negative and positive controls as described above, or there may be separate controls for each analyte being measured.

The two or more analytes differ from one another. The two or more analytes may be a macromolecule and/or a small molecule. The macromolecule is typically a protein, such as a protein-based hormone, and may also be part of a larger particle, such as a virus, a bacterium, a cell (e.g. a red blood cell) or a prion. The small molecule may be a drug.

The term “small molecule” used herein is a term of the art and is used to distinguish the molecule from macromolecules such as proteins and nucleic acids. A small molecule is often referred to in the field of immunoassays as a “hapten”, being a small molecule which, when attached to a large carrier molecule such as a protein, can elicit an immune response and includes molecules such as hormones and synthetic drugs. A small molecule of this type will typically have a molecular weight of 2,000 or less, often 1,000 or less and even 500 or less. The two or more binding components may be adapted to bind to the corresponding analyte itself, although the corresponding analyte can undergo a chemical reaction or initial complexing event before binding to the two or more binding components. For example, the two or more analytes might be protonated/deprotonated in the pH of the assay conditions. Thus, the two or more analytes which are bound to the corresponding binding component may be the analytes themselves or derivatives of the analytes; both are included within the scope of the present invention.

As well as detecting different analytes, the two or more analytes could also include different forms of the same analyte, depending upon antibody specificity. For example, proteins with post-translational modifications may contain different phosphorylation or glycosylation sites and multiplexing could be used to detect the different fractions of the target protein.

The two or more reporter reagents could also be selected based on the concentration of the two or more analytes. For example, for a low abundance analyte, a small, high affinity reporter reagent should be used to maximise the sensitivity of the assay. Under these conditions, the reporter reagent captures a higher amount of analyte and diffuses rapidly to the surface of the substrate, which maximises binding events and improves signal-to-noise. Conversely, for a high abundance analyte, a large, low affinity reporter reagent should be used to prevent saturation. The larger reporter reagent has greater surface area and diffuses to the surface of the substrate more slowly, providing less sensitivity but being able to measure to higher concentrations.

The sample which is suspected of containing the analytes of interest will generally be a fluid sample, e.g. a liquid sample, and usually a biological sample, such as a bodily fluid, e.g. blood, plasma, saliva, serum, intraocular fluid, cerebrospinal fluid or urine. The sample may contain suspended particles and may even be whole blood. In a preferred embodiment, the sample is unprocessed and more preferably, is an unprocessed fluid. By unprocessed is meant that the sample/fluid is not pre-treated by filtration, dilution or any other pre-processing step prior to being mixed with the reporter reagent and other assay components. An advantage of the method of the present invention is that the assay may be performed on a sample which contains suspended particles without unduly influencing the results of the assay.

In a preferred embodiment, the sample is whole blood. It is surprising that the components of whole blood do not interfere with the method of detection of the present invention. It is usual to remove the red blood cells from blood to measure fluorescence in the plasma or serum component of the blood due to unpredictable scattering of light by the cellular components, which varies from sample to sample. However, in the method of the present invention, because the fluorescence would be measured on the substrate using an imaging system with a shallow depth of field, and because individual binding events can be measured, the measurement can take place in whole blood.

The sample will typically be in the order of microlitres (e.g. 1-100 μL, preferably 1-10 μL). In order to hold a fluid sample, the substrate is preferably located in a sample chamber having one or more side walls, an upper surface and a lower surface. Accordingly, the device used in the method of the present invention preferably further comprises a chamber for holding the sample containing the analytes in contact with the substrate.

A potential additional source of background interference is the settling of suspended particles on to the surface of the substrate, including reporter reagent and cellular components of the sample. This source of interference may be reduced by positioning the substrate above the bulk solution, e.g. on the upper surface of the reaction chamber. Thus, if any settling occurs, it will not interfere with the substrate. Preferably the substrate forms the upper surface as shown in the figures. Preferably, the substrate is substantially planar. More preferably, the substrate is planar. By “substantially planar” it is meant that the substrate deviates from planarity only up to a point where it remains functional in the invention, for example, such that the whole of the substrate remains within a single focal range when being imaged. Clearly, the optical and two or more binding components will be on the interior surfaces of the chamber to allow contact with the sample. This and other modifications are included in the scope of the present invention.

The sample may simply be retained by surface tension forces, for example, inside a capillary channel.

The two or more reporter reagents and optionally one or more additional reagents are preferably stored in a chamber incorporated into the device used in the method of the present invention.

The method of the present invention is especially useful in point-of-care (POC) testing. POC testing is defined as diagnostic testing at or near the point of care, i.e. bedside testing. POC testing enables convenient and rapid testing, allowing improved decision making and triage whilst better allocating hospital resources such as accident and emergency care and hospital beds. This contrasts with traditional testing in which samples were taken at the point of care and then sent to the laboratory for testing. With such testing, it often takes hours or days to get the results, during which time care must continue without the desired information. POC testing often uses test kits in combination with portable instruments.

The method of the present invention is particularly useful for monitoring the concentration or presence/absence of analytes that are normally in very low abundance. Potential applications including measuring biomarkers in cardiac disease (e.g. high-sensitivity troponin), infectious disease (e.g. Hepatitis C core antigen), aging/dementia (e.g. Alzheimer's disease markers Amyloid Beta and phosphorylated Tau), cytokines and oncology (e.g. circulating tumour markers).

The present invention may also provide a device for detecting two or more analytes in a sample, the device comprising: a substrate having an optical component and two or more binding components, wherein the optical component and the two or more binding components are attached to a surface of the substrate, the optical component being capable of changing from a first optical state to a second optical state in response to interaction with irradiated photosensitisers of two or more reporter reagents bound to the surface of the substrate in proportion to the concentration of the corresponding analyte in the sample, thereby to form two or more sets of local regions having the second optical state on the substrate, wherein the two or more sets of local regions are distinguishable from one another by rate of formation of the sets of local regions, order of formation of the sets of local regions, or size of local regions optionally in combination with rate of formation of the sets of local regions or order of formation of the sets of local regions.

The features of the device are as described above for the device used in the method of the present invention.

In a preferred embodiment, the device further comprises a chamber for holding a mixture of the sample and the two or more reporter reagents.

The device may include one or more radiation sources which are adapted to generate electromagnetic radiation and a detector which is adapted to detect the second optical state thereby allowing a precise determination of the position of the photosensitiser with respect to the substrate.

The device may take the form of a cartridge to be used with a separate reader. The reader may incorporate the radiation source(s) and the detector. The reader is preferably a portable reader. Preferably, the device comprises a cartridge, wherein the substrate is within the cartridge, and wherein the device further comprises a detector for detecting the sets of local regions having the second optical state on the substrate. The present invention may also provide the cartridge comprising the substrate and the optical and binding components as defined herein. The cartridge is preferably a disposable cartridge.

The present invention may also provide a system for detecting two or more analytes in a sample, comprising: the device as described above; and two or more reporter reagents for forming a mixture comprising the sample, each of the two or more reporter reagents comprising a photosensitiser, wherein the photosensitiser is capable of absorbing electromagnetic radiation to interact with the optical component to change the optical component from the first optical state to the second optical state.

Preferably, the photosensitiser is capable of absorbing electromagnetic radiation to interact with a pre-activator reagent present in the mixture to generate an activator reagent, and the activator reagent is capable of interacting with the optical component to change the optical component from the first optical state to the second optical state.

In a preferred embodiment, the system of the present invention consists essentially of the above-described features. By “essentially” is meant that no other features are required to perform the assay.

The present invention will now be described with reference to the following examples which are not intended to be limiting. These examples demonstrate binding of the two or more reporter reagents to the corresponding binding component via the corresponding analyte. However, it is to be understood that a variety of assay formats are envisaged where the binding of the two or more reporter reagents to the corresponding binding component is either promoted or inhibited by the presence of corresponding analyte in solution. The examples are given to demonstrate the mode of conversion of the optical component from the first optical state to the second optical state.

### EXAMPLES

### Example 1

Measurement is carried out in one reaction well. A reaction well as shown in FIG. 12 can be created by taking a 20×20 mm piece of polymethyl methacrylate (PMMA) of 175 micron thickness and attaching a 1 cm by 1 cm square piece of 100 μm thickness pressure sensitive adhesive (PSA) 16 with a 6 mm diameter hole cut out to the cover slip 17 to create a shallow well 18.

The surface is first coated with biotinylated BSA, then Cy2-labelled streptavidin, using methods known in the art. The reaction well is then co-coated with two biotinylated capture antibodies, one antibody to analyte A, another antibody to analyte B. The release liner may then be removed from the PSA and the substrate inverted and attached to a piece of PMMA sheet 19 with two small holes 20 drilled through to create a reaction chamber, as shown in profile in FIG. 13.

A sample is mixed with two types of reporter reagent. The reporter reagent for analyte A is comprised of 50 nm beads coated with antibody to analyte A (photosensitiser-labelled antibody 3). The reporter reagent for analyte B is comprised of 250 nm beads coated with antibody to analyte B (photosensitiser-labelled antibody 4). Both reporter reagents are infused with the same photosensitiser that is stimulated at the same wavelength (680 nm). Photosensitiser-labelled antibody 3 generates spots of 400 nm diameter and photosensitiser-labelled antibody 4 generates spots of 1000 nm diameter.

The mixture is added to the reaction well and incubated (to allow binding) such that, as shown in FIG. 2, photosensitiser-labelled antibody 3 binds to the surface of the substrate by means of antibody 2a and photosensitiser-labelled antibody 4 binds to the surface of the substrate by means of antibody 2b. The sensor is then illuminated with light at 680 nm to activate both reporter reagents simultaneously as shown in FIG. 3. A series of large and small spots is formed on the surface as shown in FIGS. 4 and 5. The surface is then imaged by fluorescence excitation at 490 nm. The concentration of analyte A is calculated from the number of small spots and the concentration of analyte B is calculated from the number of large spots.

### Example 2

Measurement is carried out in one reaction well. The reaction well is prepared as in Example 1.

A sample is mixed with two types of reporter reagent. The reporter reagent for analyte A is comprised of 50 nm beads coated with antibody to analyte A (photosensitiser-labelled antibody 3). The reporter reagent for analyte B is comprised of 50 nm beads coated with antibody to analyte B (photosensitiser-labelled antibody 5). Photosensitiser-labelled antibody 3 is stimulated at 680 nm and photosensitiser-labelled antibody 5 is stimulated at 350 nm.

The mixture is added to the reaction well and incubated such that, as shown in FIG. 7, photosensitiser-labelled antibody 3 binds to the surface of the substrate by means of antibody 2a and photosensitiser-labelled antibody 5 binds to the surface of the substrate by means of antibody 2b (to allow binding). The sensor is illuminated first with light at 680 nm to generate spots as shown in FIGS. 8 and 9. These are then imaged and counted by fluorescence excitation at 490 nm. Subsequently, the sensor is illuminated with light at 350 nm to generate additional spots as shown in FIGS. 10 and 11. The additional spots are then counted by fluorescence excitation at 490 nm.

The concentration of analyte A is calculated from the first series of spots that formed and the concentration of analyte B is calculated from the second series of spots that formed (the total number of spots minus the first series of spots).

### Example 3

Measurement is carried out in two reaction wells. The reaction wells are prepared as in Example 1.

Reaction well 1 is coated in antibody to analyte A and reaction well 2 is coated in antibody to analyte B.

A sample is mixed with two types of reporter reagent simultaneously. The reporter reagent for analyte A is comprised of 50 nm beads coated with antibody to analyte A (photosensitiser-labelled antibody 3). The reporter reagent for analyte B is comprised of 250 nm beads coated with antibody to analyte B (photosensitiser-labelled antibody 4). Both reporter reagents are stimulated at the same wavelength (680 nm). Photosensitiser-labelled antibody 3 generates spots of 400 nm diameter, and photosensitiser-labelled antibody 4 generates spots of 1000 nm diameter.

The mixture is added to each well and allowed to incubate for 10 minutes such that, as shown in FIG. 14, photosensitiser-labelled antibody 3 binds to the surface of the substrate in well 1 by means of antibody 2a and photosensitiser-labelled antibody 4 binds to the surface of the substrate in well 2 by means of antibody 2b. After incubation (to allow binding in both wells), each well is stimulated with light at 680 nm to generate dark spots in each well as shown in FIGS. 15 and 16. These are then imaged and counted by fluorescence excitation at 490 nm.

In well 1, the number of small spots is counted to calculate the concentration of analyte A. Any large spots generated by non-specific binding of reporter B are discounted. Similarly, in well 2, the number of large spots is counted to calculate the concentration of analyte B. Any small spots generated by non-specific binding of reporter A are discounted.

Wells 1 and 2 act as negative controls for each other. In this regard, any large spots generated by non-specific binding of reporter B in well 1 acts a negative control for the large spots generated by specific binding of reporter B in well 2. The number of any large spots generated by non-specific binding of reporter B in well 1 is subtracted from the number of large spots generated by specific binding of reporter B in well 2 to provide a more accurate calculation of the concentration of analyte B.

Similarly, any small spots generated by non-specific binding of reporter A in well 2 acts a negative control for the small spots generated by specific binding of reporter A in well 1. The number of any small spots generated by non-specific binding of A in well 2 is subtracted from the number of small spots generated by specific binding of reporter A in well 1 to provide a more accurate calculation of the concentration of analyte A.

Additionally, if the non-specific binding in any well is above a threshold value, it can flag to the user that there may be an anomaly with the measurement.

### Example 4

Measurement is carried out in two reaction wells. The reaction wells are prepared as in Example 1.

Reaction well 1 is coated in antibody to analyte A and reaction well 2 is coated in antibody to analyte B.

A sample is mixed with two types of reporter reagent simultaneously. The reporter reagent for analyte A is comprised of 50 nm beads coated with antibody to analyte A (photosensitiser-labelled antibody 3). The reporter reagent for analyte B is comprised of 50 nm beads coated with antibody to B (photosensitiser-labelled antibody 5). Photosensitiser-labelled antibody 3 is stimulated at 680 nm and photosensitiser-labelled antibody 5 is stimulated at 350 nm.

The mixture is added to each well and allowed to incubate for 10 minutes such that, as shown in FIG. 17, photosensitiser-labelled antibody 3 binds to the surface of the substrate in well 1 by means of antibody 2a and photosensitiser-labelled antibody 5 binds to the surface of the substrate in well 2 by means of antibody 2b. After incubation (to allow binding in both wells), each well is stimulated with light at 680 nm to generate dark spots primarily in well 1. Each well is then imaged and the number of spots generated in well 1 is counted by fluorescence excitation at 490 nm. Subsequently, each well is stimulated with light at 350 nm to generate dark spots primarily in well 2. The number of spots generated in well 2 is then counted by fluorescence excitation at 490 nm. FIG. 18 shows well 1 being stimulated with light at 680 nm and well 2 being stimulated with light at 350 nm and FIG. 19 shows dark spots formed in both wells 1 and 2 after the illumination sequence. For simplicity, FIGS. 18 and 19 only show the formation of the specific dark spots due to specific binding. They do not include any dark spots formed due to non-specific binding.

In well 1, the number of spots is used to calculate the concentration of analyte A and similarly, the number of spots in well 2 is used to calculate the concentration of analyte B.

Wells 1 and 2 act as negative controls for each other. In this regard, any spots generated by non-specific binding of reporter B in well 1 acts a negative control for the spots generated by specific binding of reporter B in well 2. The number of any spots generated by non-specific binding of reporter B in well 1 is subtracted from the number of spots generated by specific binding of reporter B in well 2 to provide a more accurate calculation of the concentration of analyte B.

Similarly, any spots generated by non-specific binding of reporter A in well 2 acts a negative control for the spots generated by specific binding of reporter A in well 1. The number of any spots generated by non-specific binding of A in well 2 is subtracted from the number of spots generated by specific binding of reporter A in well 1 to provide a more accurate calculation of the concentration of analyte A.

1. A method for detecting two or more analytes in a sample, the method comprising the steps of:
(i) providing a mixture comprising a sample and two or more reporter reagents to a device, wherein each of the two or more reporter reagents comprises a photosensitiser, the device comprising a substrate having an optical component and two or more binding components, wherein the optical component and the two or more binding components are attached to a surface of the substrate;
(ii) allowing a portion of each of the two or more reporter reagents to bind to the surface of the substrate in proportion to the concentration of the corresponding analyte, by means of the corresponding binding component;
(iii) irradiating the device with electromagnetic radiation at one wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) optionally detecting the at least one set of local regions of the optical component having the second optical state on the substrate formed in step (iii) and repeating step (iii) with electromagnetic radiation at the same or a different wavelength for absorption by at least one of the photosensitisers of the two or more reporter reagents, such that at least one of the photosensitisers of the two or more reporter reagents bound to the surface of the substrate absorbs electromagnetic radiation and interacts with the optical component to cause the optical component to change from the first optical state to the second optical state, thereby forming at least one set of local regions of the optical component having the second optical state on the substrate; and
(v) if step (iv) is performed, detecting the at least one set of local regions of the optical component having the second optical state on the substrate formed in step (iv) or, if step (iv) is not performed, detecting two or more sets of local regions having the second optical state on the substrate formed in step (iii),
wherein the two or more sets of local regions are distinguishable from one another by rate of formation of the sets of local regions, order of formation of the sets of local regions, or size of local regions optionally in combination with rate of formation of the sets of local regions or order of formation of the sets of local regions.

2. The method as claimed in claim 1, wherein the two or more reporter reagents differ from one another by size, gas permeability, excitation wavelength of the photosensitiser, reactivity of the photosensitiser, amount of the photosensitiser or combinations thereof.

3. The method as claimed in claim 1, wherein step (iii) is
irradiating the device with electromagnetic radiation at one wavelength for absorption by the photosensitiser of each of the two or more reporter reagents, such that the photosensitiser of each of the two or more reporter reagents bound to the surface of the substrate absorbs electromagnetic radiation and interacts with the optical component to cause the optical component to change from the first optical state to the second optical state, thereby forming two or more sets of local regions of the optical component having the second optical state on the substrate;
step (iv) is not performed; and
in step (v), the two or more sets of local regions are distinguishable from one another by size of local regions.

4. The method as claimed in claim 3, wherein the two or more reporter reagents differ from one another by size, gas permeability, reactivity of the photosensitiser, amount of the photosensitiser or combinations thereof.

5. The method as claimed in claim 1, wherein step (iv) is performed; and
In step (v), the two or more sets of local regions are distinguishable from one another by rate of formation of the sets of local regions, order of formation of the sets of local regions, or size of local regions in combination with rate of formation of the sets of local regions or order of formation of the sets of local regions.

6. The method as claimed in claim 1, wherein each of the two or more binding components is attached to a separate area of the surface of the substrate.

7. The method as claimed in claim 1, wherein each of the two or more reporter reagents further comprises a polymer particle and the photosensitiser is encapsulated within the polymer particle.

8. The method as claimed in claim 1, wherein the photosensitiser of each of the two or more reporter reagents absorbs electromagnetic radiation and interacts with a pre-activator reagent present in the mixture to generate an activator reagent which interacts with the optical component to cause the optical component to change from the first optical state to the second optical state.

9. The method as claimed in claim 8 wherein the activator reagent is a reactive oxygen species.

10. The method as claimed in claimed in claim 9, wherein the reactive oxygen species is singlet oxygen.

11. The method as claimed in claim 1, wherein the first optical state is fluorescent and the second optical state is non-fluorescent.

12. The method as claimed in claim 1, wherein the change from the first optical state to the second optical state is irreversible.

13. The method as claimed in claim 1, wherein steps (i) to (iii) take place in the absence of wash steps.

14. The method as claimed in claim 1, wherein the sample is unprocessed.

15. The method as claimed in claim 1, wherein the device is irradiated with electromagnetic radiation for more than 1 second.
